(19) United States
(12) Patent Application Publication
(10) Pub. No.: US 2025/0161030 A1
(43) Pub. Date: May 22, 2025

(54) PROSTHETIC HEART VALVE INCLUDING LEAFLET ABRASION PROTECTION

(71) Applicant: MEDTRONIC, INC., Minneapolis, MN (US)

(72) Inventors: Alkindi Kibria, Irvine, CA (US); Philip C. Leung, Midway City, CA (US); Karl L. Olney, irvine, CA (US); Kshitija P. Garde, Fullerton, CA (US); Kelly T Tran, Garden Grove, CA (US)

(21) Appl. No.: 18/728,900

(22) PCT Filed: Jan. 18, 2023
(86) PCT No.: PCT/IB2023/050420

§ 371 Date: Jul. 15, 2024

Related U.S. Application Data
(60) Provisional application No. 63/600,281, filed on Nov. 17, 2023.
(60) Provisional application No. 63/345,237, filed on May 24, 2022.

(51) Int. Cl. A61F 2/24

(52) U.S. Cl. CPC A61F 2/2418; A61F 2230/0069; A61F 2250/001

(57) ABSTRACT

A prosthetic heart valve includes an annular stent having an inflow portion comprising a plurality of rows of angled inflow struts, an outflow portion comprising a row of angled outflow struts, and a transition portion comprising an axial strut and a commissure post, a prosthetic valve including a leaflet structure positioned within the stent and secured to the at least one commissure post, and a tissue bumper configured to reduce abrasion caused by contact by the leaflet structure with the annular stent. The tissue bumper comprises a tissue strip wrapped around at least one angled outflow strut in the row of angled outflow struts, the axial strut, and a portion of a first row of angled struts of the plurality of rows of angled inflow struts.

### FIELD

The present technology is generally related to medical devices, and, more particularly, to prosthetic heart valves.

### BACKGROUND

Patients suffering from various medical conditions or diseases may require surgery to install an implantable medical device. For example, valve regurgitation or stenotic calcification of leaflets of a heart valve may be treated with a heart valve replacement procedure. A traditional surgical valve replacement procedure requires a sternotomy and a cardiopulmonary bypass, which creates significant patient trauma and discomfort. Traditional surgical valve procedures may also require extensive recuperation times and may result in life-threatening complications.

One alternative to a traditional surgical valve replacement procedure is delivering implantable medical devices using minimally-invasive techniques. For example, a prosthetic heart valve can be percutaneously and transluminally delivered to an implant location. In such methods, the prosthetic heart valve can be compressed or crimped on a delivery catheter for insertion within a patient's vasculature; advanced to the implant location; and re-expanded to be deployed at the implant location.

It is desirable to maximize the effective orifice area of prosthetic heart valves, that is, the leaflet opening. However, in some instances, maximizing effective orifice area and other factors may lead to leaflet-to-frame and/or leaflet-to-skirt contact, which may cause leaflet abrasion, resulting in reduced durability of the leaflets.

### SUMMARY

The techniques of this disclosure generally relate to frames or stents for implantable medical devices that include tissue bumpers.

In a first aspect, the present disclosure provides a prosthetic heart valve that is radially collapsible to a collapsed configuration and radially expandable to an expanded configuration. The prosthetic heart valve includes an annular stent having a longitudinal axis extending between an inflow end of the stent and an outflow end of the annular stent. The annular stent includes an inflow portion comprising a plurality of rows of angled inflow struts, an outflow portion comprising a row of angled outflow struts, and a transition portion comprising an axial strut and a commissure post. The prosthetic heart valve further includes a prosthetic valve including a leaflet structure positioned within the stent and secured to the at least one commissure post. The prosthetic heart valve further includes a tissue bumper configured to reduce abrasion caused by contact by the leaflet structure with the annular stent, the tissue bumper comprising a tissue strip wrapped around at least one angled outflow strut in the row of angled outflow struts, the axial strut, and a portion of a first row of angled struts of the plurality of rows of angled inflow struts.

In a second aspect, in the prosthetic heart valve of the first aspect, a center portion of the tissue strip is wrapped around an outflow end of the commissure post, a first end portion the tissue strip extends from the commissure post in a first direction and is wrapped around outflow struts of the row of angled outflow struts to the axial strut, the first end portion extends along and is wrapped around the axial strut toward the inflow end of the annular stent, and the first end portion extends in a second direction opposite the first direction from an inflow end of the axial strut and wraps around angled inflow struts of the first row of angled inflow struts.

In a third aspect, in the prosthetic heart valve of the second aspect, the first end portion of the tissue strip extends along at least two angled inflow struts of the first row of angled inflow struts.

In a fourth aspect, in the prosthetic heart valve of the second aspect or the third aspect, a second end portion the tissue strip extends from the commissure post in the second direction and is wrapped around outflow struts of the row of angled outflow struts to a second axial strut disposed on an opposite side of the commissure post as the axial strut, the second end portion extends along and is wrapped around the second axial strut toward the inflow end of the annular stent, and the second end portion extends in the first direction from an inflow end of the second axial strut and wraps around angled inflow struts of the first row of angled inflow struts.

In a fifth aspect, in the prosthetic heart valve of any of the second through fourth aspects, The prosthetic heart valve of claim 3, wherein the first end portion of the tissue strip extends longitudinally towards the inflow end of the annular stent to a first end of the tissue strip.

In a sixth aspect, in the prosthetic heart valve of the fifth aspect, a second end portion the tissue strip extends from the commissure post in the second direction and is wrapped around outflow struts of the row of angled outflow struts to a second axial strut disposed on an opposite side of the commissure post as the axial strut, the second end portion extends along and is wrapped around the second axial strut toward the inflow end of the annular stent, the second end portion extends in the first direction from an inflow end of the second axial strut and wraps around angled inflow struts of the first row of angled inflow struts, and the second end portion of the tissue strip extends longitudinally towards the inflow end of the annular stent to a second end of the tissue strip.

In a seventh aspect, the prosthetic heart valve of the second through sixth aspects further includes a skirt coupled to the inflow portion of the annular stent, wherein the first and second ends of the tissue bumper are coupled to an exterior surface of the skirt.

In an eighth aspect, in the prosthetic heart valve of the second or third aspects, the first end portion of the tissue strip extends generally parallel to an angled inflow strut of a second row of the plurality of rows of angled inflow struts to a first end of the tissue strip generally axially aligned with the commissure post.

In a ninth aspect, in the heart valve prosthesis of the eighth aspect, a second end portion the tissue strip extends from the commissure post in the second direction and is wrapped around outflow struts of the row of angled outflow struts to a second axial strut disposed on an opposite side of the commissure post as the axial strut, the second end portion extends along and is wrapped around the second axial strut toward the inflow end of the annular stent, the second end portion extends in the first direction from an inflow end of the second axial strut and wraps around angled inflow struts of the first row of angled inflow struts, and the second end portion of the tissue strip extends generally parallel to an angled inflow strut of a second row of the plurality of rows of angled inflow struts to a second end of the tissue strip generally axially aligned with the commissure post.

In a tenth aspect, the heart valve prosthesis of the ninth aspect further includes a skirt coupled to the inflow portion of the annular stent, wherein the first and second ends of the tissue strip are coupled to an exterior surface of the skirt.

In an eleventh aspect, in the heart valve prosthesis of the ninth aspect, the first and second ends of the tissue strip are attached to each other.

In a twelfth aspect, in the heart valve prosthesis of the third aspect, the first end portion of the tissue strip extends generally circumferentially to a first end disposed generally at a center of a cell defined by the first row and a second row of the plurality an angled inflow struts, wherein the center of the cell is axially aligned with the commissure post.

In a thirteenth aspect, in heart valve prosthesis of the twelfth aspect, a second end portion the tissue strip extends from the commissure post in the second direction and is wrapped around outflow struts of the row of angled outflow struts to a second axial strut disposed on an opposite side of the commissure post as the axial strut, the second end portion extends along and is wrapped around the second axial strut toward the inflow end of the annular stent, the second end portion extends in the first direction from an inflow end of the second axial strut and wraps around angled inflow struts of the first row of angled inflow struts, and the second end portion of the tissue strip extends generally circumferentially to a first end disposed generally at the center of the cell.

In a fourteenth aspect, the heart valve prosthesis of the any of the previous aspects further includes a skirt coupled to the inflow portion of the annular stent, wherein the first and second ends of the tissue strip are coupled to an exterior surface of the skirt.

In a fifteenth aspect, in the prosthetic heart valve any of the previous aspects, the first and second ends of the tissue strip are attached to each other.

In a sixteenth aspect, in the prosthetic heart valve of any of the previous aspects, the annular stent further includes a second commissure post and a second tissue bumper comprising a second tissue strip, wherein a first end portion of the second tissue strip overlaps with the second end portion of the tissue strip of the tissue bumper along the second axial strut.

In a seventeenth aspect, the present disclosure provides a prosthetic heart valve that is radially collapsible to a collapsed configuration and radially expandable to an expanded configuration. The prosthetic heart valve includes an annular stent having a longitudinal axis extending between an inflow end of the stent and an outflow end of the annular stent. The annular stent includes an inflow portion comprising a plurality of rows of angled inflow struts, an outflow portion comprising a row of angled outflow struts, and a transition portion comprising an axial strut and a commissure post. The prosthetic heart valve further includes a prosthetic valve comprising a leaflet structure positioned within the stent and secured to the commissure post. The prosthetic heart valve further includes a tissue bumper configured to reduce abrasion caused by contact by the leaflet structure with the annular stent, the tissue bumper comprising a tissue strip coupled to the axial strut and a portion of a first row of angled inflow struts of the plurality of rows of angled inflow struts.

In an eighteenth aspect, in the prosthetic heart valve of the seventeenth aspect, a central portion of the tissue strip is wrapped around an inflow end the axial strut, a first end portion of the tissue strip extends in a first direction from the axial strut and is wrapped around a plurality of angled inflow struts of the first row of angled inflow struts, and a second end portion of the tissue strip extends in a second direction opposite the first direction from the axial strut and is wrapped around a second plurality of angled inflow struts of the first row of angled inflow struts.

In a nineteenth aspect, in the prosthetic heart valve of the seventeenth aspect, a central portion of the tissue strip is wrapped around an outflow end the axial strut, a first end portion of the tissue strip and a second end portion are wrapped around the axial strut towards in inflow end of the axial strut, the first end portion extends in a first direction from the axial strut and is wrapped around a plurality of angled inflow struts of the first row of angle inflow struts, and a second end portion of the tissue strip extends in a second direction opposite the first direction from the axial strut and is wrapped around a second plurality of angled inflow struts of the first row of angled inflow struts.

In a twentieth aspect, in prosthetic heart valve of the eighteenth or nineteenth aspects, the first end portion of the tissue strip extends generally parallel to an angled inflow strut of a second row of the plurality of rows of angled inflow struts to a first end of the tissue strip generally axially aligned with the commissure post, and the second end portion of the tissue strip extends generally parallel to an angled inflow strut of a second row of the plurality of rows of angled inflow struts to a second end of the tissue strip generally axially aligned with a second commissure post.

In a twenty-first aspect, the prosthetic heart valve of the twentieth aspect further includes a second tissue strip coupled to a second axial strut, wherein the second tissue strip includes a first end portion extending generally parallel to a second angled inflow strut of the second row of the plurality of rows of angled inflow struts to a first end of the second tissue strip generally axially aligned with the second commissure post.

In a twenty-second aspect, in the prosthetic heart valve of the eighteenth aspect or the nineteenth aspect, the first end portion of the tissue strip extends generally circumferentially to a first end disposed generally at a center of a cell defined by the first row and a second row of the plurality an angled inflow struts, wherein the center of the cell is axially aligned with the commissure post, and the second end portion of the tissue strip extends generally circumferentially to a second end disposed generally at a center of a second cell defined by the first row and the second row of the plurality an angled inflow struts, wherein the center of the second cell is axially aligned with a second commissure post.

In a twenty-third aspect, the prosthetic heart valve of the twenty-second aspect further includes a second tissue strip coupled to a second axial strut, wherein the second tissue strip includes a first end portion extending generally circumferentially to a first end disposed generally at the center of the second cell adjacent the second end of the first tissue strip.

In a twenty-fourth aspect, in the prosthetic heart valve of any one of the nineteenth through twenty-third aspects, the first and second end portions of the tissue strip are wrapped around the axial strut in an overlay wrap.

In a twenty-fifth aspect, in the prosthetic heart valve of any one of the nineteenth through twenty-third aspects the first and second end portions of the tissue strip are wrapped around the axial strut in a barber pole wrap.

In a twenty-sixth aspect, in the prosthetic heart valve of any one of the nineteenth through twenty-third aspects, wherein the first and second end portions of the tissue strip are wrapped around the axial strut in a weave wrap.

In a twenty-seventh aspect, the prosthetic heart valve of any one of the nineteenth through twenty-sixth aspects further includes a skirt coupled to the inflow portion of the stent.

In a twenty-eighth aspect, in the prosthetic heart valve of the twenty-seventh aspect, the skirt includes holes positioned adjacent to the portion of angled inflow struts in the first row of the plurality of angled inflow struts, and the tissue strip passes through the holes as it is wrapped around the portion of the first row angled inflow struts.

In a twenty-ninth aspect, in the prosthetic heart valve of any one of the twenty-first or twenty-third aspects, the second end of the first tissue trip is attached to the first end of the second tissue strip.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings are not to scale.

FIGS. 1A-1C depict illustrations of a prosthetic heart valve which include markers, according to an embodiment hereof.

FIGS. 2A-2D depict illustrations of tissue bumpers attached to a stent of a prosthetic heart valve, according to an embodiment hereof.

FIGS. 3A-3C depict illustrations of tissue bumpers attached to a stent of a prosthetic heart valve, according to an embodiment hereof.

FIGS. 4A-4C depict illustrations of tissue bumpers attached to a stent of a prosthetic heart valve, according to an embodiment hereof.

FIGS. 5A-5C depict illustrations of tissue bumpers attached to a stent of a prosthetic heart valve, according to an embodiment hereof.

FIGS. 6A-6C depict illustrations of tissue bumpers attached to a stent of a prosthetic heart valve, according to an embodiment hereof.

FIGS. 7A-7C depict illustrations of tissue bumpers attached to a stent of a prosthetic heart valve, according to an embodiment hereof.

FIGS. 8A-8B depict illustrations of tissue bumpers attached to a stent of a prosthetic heart valve, according to an embodiment hereof.

FIGS. 9A-9B depict illustrations of tissue bumpers attached to a stent of a prosthetic heart valve, according to an embodiment hereof.

FIGS. 10A-10D depict illustrations of tissue bumpers attached to a stent of a prosthetic heart valve, according to an embodiment hereof.

FIGS. 11A-11C depict illustrations of tissue bumpers attached to a stent of a prosthetic heart valve, according to an embodiment hereof.

FIGS. 12A-12B depict illustration of an overlay wrap for wrapping a portion of a tissue bumper around an axial strut of a stent, according to embodiments hereof.

### DETAILED DESCRIPTION

Specific embodiments of the present disclosure are now described with reference to the figures. The following detailed description describes examples of embodiments and is not intended to limit the present technology or the application and uses of the present technology. Although the description of embodiments hereof is in the context of an implantable medical device, e.g., prosthetic heart valve, the present technology may also be used in other devices. Furthermore, there is no intention to be bound by any expressed or implied theory presented in the preceding technical field, background, brief summary or the following detailed description.

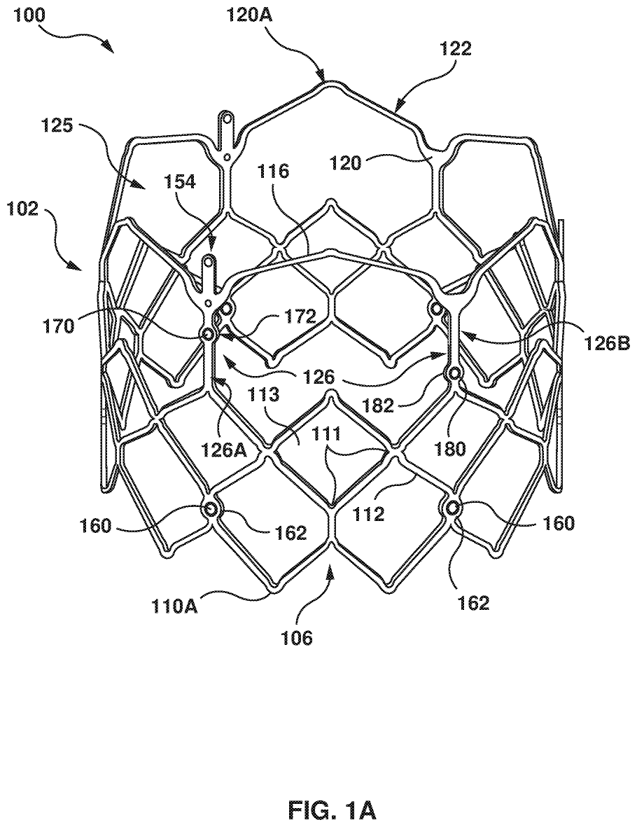

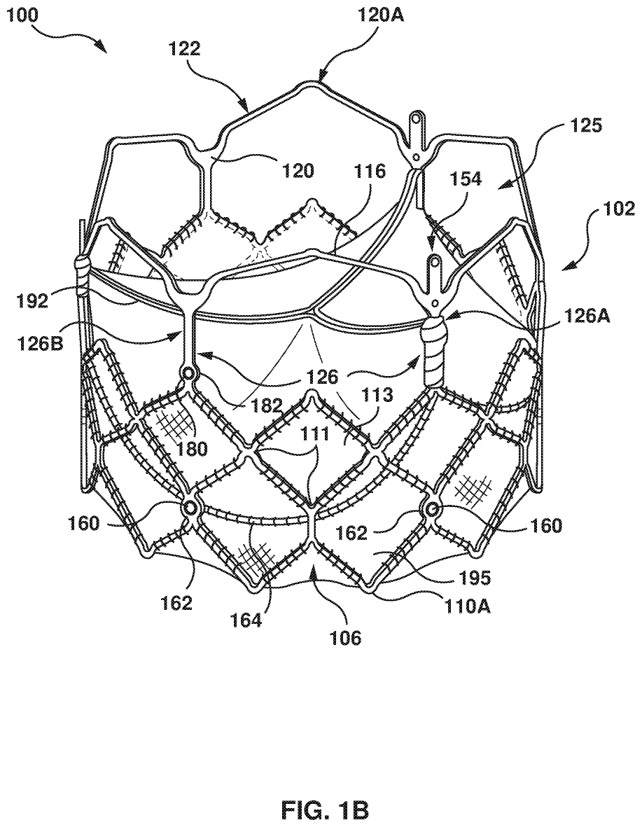

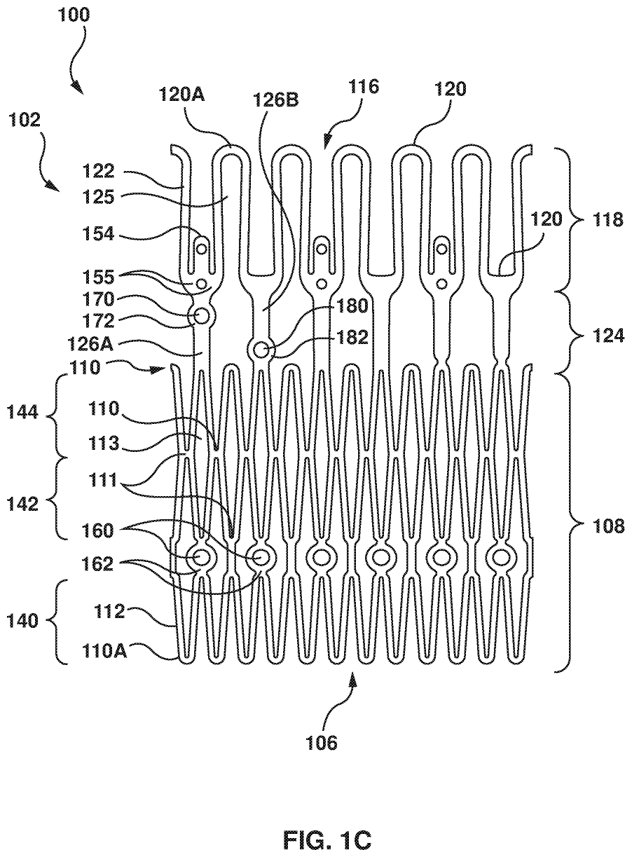

FIGS. 1A-1C illustrate a transcatheter heart valve prosthesis 100 in which a radially-expandable frame or stent 102 thereof includes leaflet abrasion protection, according to embodiment hereof One skilled in the art will realize that FIGS. 1A-1C illustrate one example of a transcatheter heart valve prosthesis and that existing components illustrated in FIGS. 1A-1C may be removed and/or additional components may be added.

The stent 102 has an expanded configuration, which is shown FIG. 1A, and a non-expanded or crimped configuration, which is not illustrated. “Non-expanded” or “crimped” configuration as used herein refers to the configuration of the stent 102 after crimping. For example, the stent 102 can be crimped onto a balloon of a balloon catheter for delivery. In some embodiments, the stent 102 can be mechanically or balloon expandable. As such, the stent 102 can be made from a plastically deformable material such that, when expanded by a dilatation balloon, the stent 102 maintains its radially expanded configuration after balloon deflation. The stent 102 can be formed from stainless steel or other suitable metal, such as platinum iridium, cobalt chromium alloys such as MP35N, or various types of polymers or other materials known to those skilled in the art, including said materials coated with various surface deposits to improve clinical functionality. In some embodiments, the stent 102 can be self-expanding. As illustrated in FIG. 1B and discussed below in further detail, a prosthetic valve 190 is disposed within and secured to the stent 102.

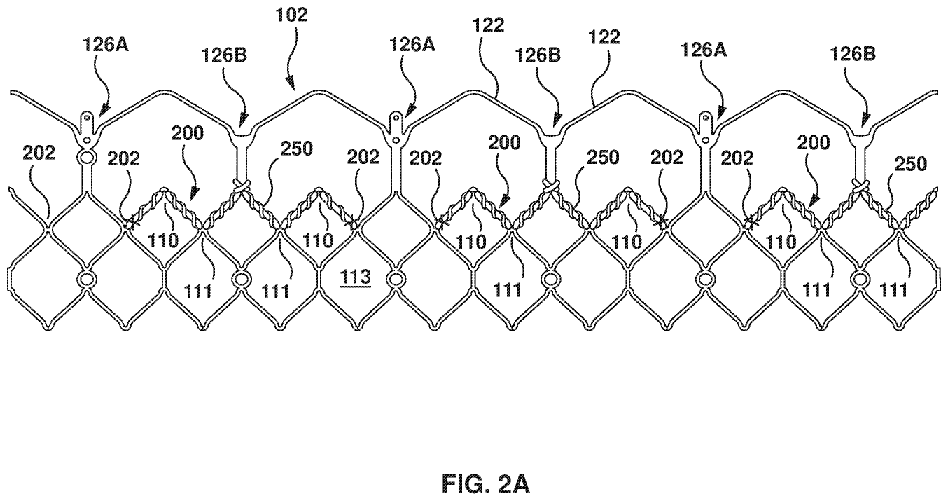

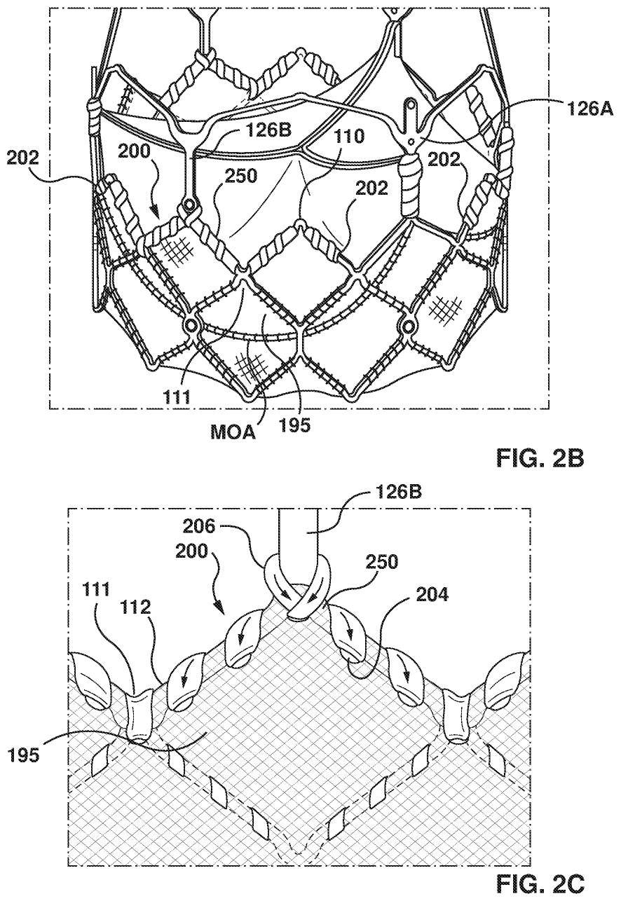

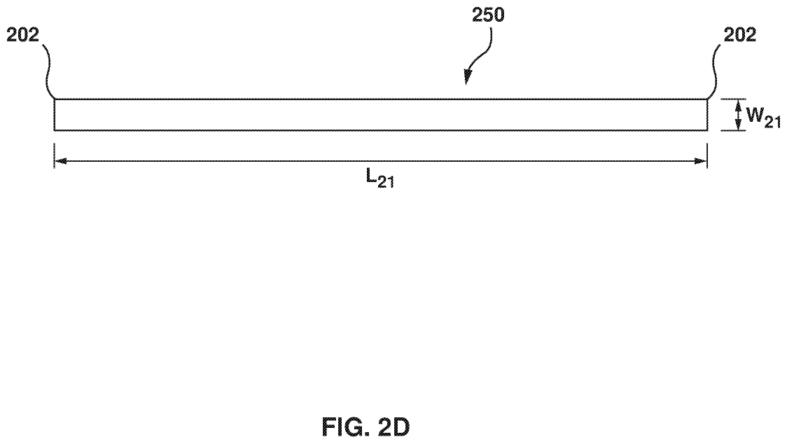

As described below in further detail with reference to FIGS. 2A-2D, 3A-3C, 4A-4C, 5A-5C, 6A-6C, 7A-7C, 8A-8B, 9A-9C, 10A-10D, and 11A-11C, the stent 102 operates to maximize effective orifice area (EOA). This EOA is directly related to maximizing the opening of leaflets of the prosthetic valve 190. However, due to the frame design (cylindrical) and limitations of balloon deployment method (e.g., expanding the valve cylindrically), maximizing the leaflet opening may cause a risk of leaflet-frame or leaflet-fabric skirt contact. This contact may cause leaflet abrasion resulting in a reduction in durability and/or increased wear on the leaflets. According to embodiments disclosed herein, a tissue strip is wrapped around portions of the stent 102 (e.g., inflow struts) to form a tissue bumper. The tissue bumper may be formed in areas where the leaflets contact the frame and/or the skirt to reduce the abrasion and allow for a valve design that can open fuller to maximize the EOA without unduly increasing wear/reducing longevity. Multiple bumpers may be used for a particular stent, with each bumper being formed by a tissue strip. Because the lubricous properties of the tissue strip, the tissue bumper provides the abrasion reduction and provides a cushioning effect. Further, as disclosed herein, multiple tissue bumpers may be used on a stent.

FIG. 1C shows a simplified open, flat view of an example of the stent 102. In embodiments, as illustrated in FIG. 1C, the stent 102 includes an inflow portion 108, an outflow portion 118, and a transition portion 124 bridging, connecting, or otherwise extending between the inflow portion 108 and the outflow portion 118. As illustrated in FIGS. 1A and 1B, the stent 102 can be a generally tubular component defining a central lumen or passageway and can have an inflow or proximal end 106 and an outflow or distal end 116. In some embodiments, when expanded, a diameter of the inflow end 106 of the stent 102 can be the same as a diameter of the outflow end 116 of the stent 102. The stent 102 can be formed by a laser-cut manufacturing method and/or another conventional stent forming method as would be understood by one of ordinary skill in the art. The lateral cross-section of the stent 102 can be trapezoidal, circular, ellipsoidal, rectangular, hexagonal, square, or other polygonal shape, although at present it is believed that trapezoidal, circular or ellipsoidal may be preferable when utilized with the replacement of an aortic valve. The stent 102 can be configured to be rigid such that it does not deflect or move when subjected to in-vivo forces, or such that deflection or movement is minimized when subjected to in-vivo forces.

As illustrated in FIGS. 1B and 1C, the prosthetic valve 190 is disposed within and secured to at least the transition portion 124 of the stent 102. In addition, the prosthetic valve 190 can also be disposed within and secured to the inflow portion 108 of the stent 102 and/or the outflow portion 118 of the stent 102. One skilled in the art will realize that the prosthetic valve 190 can be disposed within and secured to one or more of the inflow portion 108, outflow portion 118, or the transition portion 124, for example, depending on the design and construction of the prosthetic valve 190 and/or the design and construction of the stent 102.

The inflow portion 108 can be formed proximate to the inflow end 106 of the stent 102. The inflow portion 108 of the stent 102 may be formed with rows of struts 112 and bends or crowns 110 connecting circumferentially adjacent struts 112 of each row to one another. The struts 112 of the inflow portion 108 may also be referred to as inflow struts 112, angled struts 112, and/or angled inflow struts 112. The inflow struts 112 are angled with respect to the central longitudinal axis of the stent 102 and angled with respect to the circumference of the stent 102. Crowns 110 may also be referred to as inflow crowns. Nodes 111 are formed where a crown 110 of one row of struts 112 and crowns 110 joins a crown 110 of a longitudinally adjacent row of struts 112 and crowns 110. In some embodiments, the inflow end 106 of the stent 102 can include a total of twelve endmost inflow crowns 110A. Pairs of the struts 112 in a row and a pair of struts 112 in an adjacent row, coupled at the nodes 111, form a cell 113 that defines an open space in the inflow portion 108 of the stent 102.

The outflow portion 118 can be formed proximate to the outflow end 116 of the stent 102. The outflow portion 118 can be configured in a shape that forms a central lumen or passageway, for example, a ring. The outflow portion 118 can include a plurality of crowns 120 and a plurality of struts 122 with each crown 120 being formed between a pair of opposing struts 122. Each crown 120 can be a curved segment or bend extending between opposing struts 122. The struts 122 of the outflow portion 118 may also be referred to as outflow struts 122, angled struts 122, and/or angled outflow struts 122. The outflow struts 122 are angled with respect to the central longitudinal axis of the stent 102 and with respect to a circumference of the stent 102. A series of endmost outflow crowns 120A are formed at the outflow end 116 of the stent 102. In some embodiments, the outflow end 116 of the stent 102 can have a total of six endmost outflow crowns 120A.

The transition portion 124 bridges, connects, or otherwise extends between the inflow portion 108 and the outflow portion 118. In some embodiments, the transition portion 124 can include a minimum of three axial frame members (or axial struts) 126, each axial frame member 126 extending between a crown 120 of the outflow portion 118 and a crown 110 of the inflow portion 108. Each axial frame member 126 can be connected to a crown 120 of the outflow portion 118 and connected to a crown 110 of the inflow portion 108. The axial frame members 126 can be substantially parallel to the central longitudinal axis of the stent 102. Each axial frame member 126 can be disposed approximately halfway between a pair of adjacent endmost outflow crowns 120A. While the stent 124 has been described as including a transition portion 124, one skilled in the art will realize that the transition portion 124 may form a portion of the inflow portion 108 and/or the outflow portion 118

In an embodiment, the transition portion 124 can include up to six axial frame members 126, with three of the axial frame members 126 being commissure posts 126A and three of the axial frame members 126 being axial struts 126B. The commissure posts 126A and the axial struts 126B can be being alternatingly positioned, as illustrated, for example, in FIGS. 1A and 1C. The commissure posts 126A can be circumferentially spaced apart and aligned with and attached to a respective commissure of three leaflets of the prosthetic valve, and the axial struts 126B can be disposed between adjacent commissure posts 126A. The axial frame members 126 may aid in valve alignment and coaptation. More particularly, the axial frame members 126 reinforce or strengthen the commissure region of the prosthetic valve by shaping the leaflets and supporting the leaflets during opening and closing thereof, and thus provide more reliable leaflet coaptation. In addition, the axial frame members 126 maximize symmetrical cell expansion.

In an embodiment, the endmost outflow crowns 120A are not connected to the axial frame members 126 but rather may be considered to be free or unattached while the remaining outflow crowns 120 of the outflow portion 118 are connected to the axial frame members 126 and disposed closer to the inflow end 106 than the endmost outflow crowns 120A. In the embodiment shown, the stent 102 includes a single row of struts 122 and crowns 120 coupled to the axial frame members 126 and defining the outflow end 116 of the stent 102. Further, in the embodiment shown, exactly two struts 122 and a single crown 120 of the outflow portion 118 are disposed between adjacent axial frame members 126. Such an arrangement can provide a series of six endmost cells 125 formed at the outflow portion 118 of the stent 102. However, this is not meant to be limiting, and different arrangements may be utilized.

In embodiments, each endmost cell 125 can define an open space in the stent 102, which is formed in any type of shape, in the radially expanded configuration, for example, as shown in FIG. 1A. Each endmost cell 125 can be defined by two adjacent struts 122 of the outflow portion 118, four adjacent struts 112 of the inflow portion 108, and two adjacent axial frame members 126 of the transition portion 124. The endmost cells 125 of the outflow portion 118 can be relatively larger than the cells 113 of the inflow portion 108 to improve access to the coronary arteries when used for aortic valve replacement. More particularly, the endmost cells 125 of the outflow portion 118 can be configured to be of sufficient size to be easily crossed with a coronary guide catheter into either the right coronary artery or the left main coronary artery once the transcatheter valve prosthesis 100 is deployed, in situ, e.g., for a post-implantation percutaneous coronary intervention (PCI).

In an embodiment, the inflow portion 108 can include exactly three rows of struts 112 and crowns 110 between the axial frame members 126 and the inflow end 106 of the stent 102, thereby forming two rows of cells 113 in the inflow portion 108. Further, in this embodiment, the four struts 112 and three crowns 110 can be disposed circumferentially between adjacent axial frame members 126. One skilled in the art will realize that the above configuration of the inflow portion 108 is one example of a configuration of the inflow portion 108 and that the inflow portion 108 can include fewer or additional rows of struts 112 and crowns 110. Likewise, one skilled in the art will realize that each row can include fewer or additional numbers of struts 112 and crowns 110.

In an embodiment, a height or length of the stent 102 in the expanded configuration can be between 14 and 23 mm, the height being measured from the most proximal part thereof to the most distal part thereof, and a diameter of the stent 102 in the expanded configuration can be between 18 and 31 mm. For example, an expanded 21 mm diameter device would be 15 mm in height. An expanded 30 mm diameter device would have a 21 mm height. Additionally, one skilled in the art will realize that the ranges of the height and diameter of the stent 102 are examples and that the height and diameter of the stent 102 may vary based on an amount of expansion of the stent 102, for example, as required by a given application and/or governed by the design and construction of the stent 102.

In an embodiment, the axial frame members 126 can include commissure posts 126A that are formed to have an axial length greater than the axial struts 126B. In this embodiment, a first end of each of the axial struts 126B, which is closer to the inflow end 106, can be coupled to a pair of struts 112 of the inflow portion 108 or a crown 110 of the inflow portion 108. A second end of each of the axial struts 126B, which is closer to the outflow end 116, can be coupled to a pair of the struts 122 of the outflow portion 118. A first end of each of the commissure posts 126A, which is closer to the inflow end 106, can be coupled to a pair of the struts 112 of the inflow portion or a crown 110 of the inflow portion. Because the commissure posts 126A are longer than the axial struts 126B, pairs of struts 122 of the outflow portion 118 are coupled to the commissure posts 126A at side portions 155 of the commissure posts 126A (e.g., an intersection of the commissure posts 126A and struts 122). The location of the connection to the side portions 155 is spaced a distance, in the direction of the inflow end 106, from the second end of the commissure posts 126A, which is closer to the outflow end 116. In other words, each commissure posts 126A can be a relatively stiff, axial segment or planar bar having the first end connected to a pair of struts 112 at a crown 110 of the inflow portion 108 and having an unattached or free second end distal of the side portions 155. As such, the connection of the struts 122 to the side portions 155 defines an outflow portion 154 of each commissure post 126A, which is positioned in the outflow portion 118.

The outflow portions 154 can be configured as support features that allow for lengthened commissure posts 126A to further reinforce or strengthen the commissure region of the transcatheter valve prosthesis 100. Each of the outflow portions 154 can extend into the outflow portion 118 of the stent 102 to allow for lengthened commissure posts 126A without increasing the overall height of the transcatheter valve prosthesis 100. In an embodiment, the stent 102 can include a total of three commissure posts 126A, which include three outflow portions 154. The commissure posts 126A, which include the outflow portions 154, one for each commissure post 126A, can extend substantially parallel to the central longitudinal axis of the stent 102 and are circumferentially spaced apart from each other. The commissure posts 126A, which include the outflow portions 154, can include holes or openings 158 formed therein configured to attach a respective commissure of the three leaflets of the prosthetic valve to the stent 102. Additionally, in some embodiments, the commissure posts 126A, which include the outflow portions 154, can include one or more holes or openings to support alignment markers, as described further below. One skilled in the art will realize that the above configuration of the outflow portions 154 is one example of a configuration and that the stent 102 can include additional outflow portions 154 based on the design or configuration of the stent 102.

As discussed above, the commissure posts 126A can be formed to be lengthened relative to the axial struts 126B. The commissure posts 126A can reduce stresses observed at the commissure region during valve loading by spreading out such stresses across a larger area. More particularly, as compared to self-expanding valve stents, balloon expandable valves stents are stiffer and stronger but therefore may place more stress on the valve leaflets attached thereto attached to the stent 102. The valve leaflets, which are often formed from tissue, are more durable when the portion of the stent to which they are attached is more flexible, but such stent flexibility may be detrimental to stent fatigue. As such, the commissure posts 126A achieve a balance between stent durability and tissue durability because the stent 102 maintains its strength and durability while the lengthened commissure supports improve or increase tissue durability of the valve leaflets by stress relief due to the lengthened commissure supports.

Further, the performance of the transcatheter valve prosthesis 100 may be enhanced by the lengthened commissure posts 126A without increasing the overall height of the transcatheter valve prosthesis 100. For example, in the unexpanded or compressed configuration, as illustrated in FIG. 1A, the outflow portions 154 of the commissure posts 126A extend into the outflow portion 118, but do not extend beyond the endmost outflow crowns 120A. In the expanded or uncompressed configuration, as illustrated in FIG. 1C, the outflow portions 154 of the commissure posts 126A extend into the outflow portion 118, but do not extend beyond the endmost outflow crowns 120A. In other words, the length of the commissure post 126A is increased without increasing the length of the transition portion 124 and the overall height of the transcatheter valve prosthesis 100. A relatively short or minimized overall height can be desirable to increase coronary access and improve system deliverability. In another embodiment hereof (not shown), the stent 100 can include additional commissure posts 126A, which include the outflow portions 154. Inclusion of additional commissure posts 126A, which include the outflow portions 154, may aid in valve alignment and coaptation.

In embodiments, to ensure the proper placement in the native anatomy of a subject, the transcatheter valve prosthesis 100 can include inflow markers 160 that are located in openings or containment members 162 in the inflow portion 108 and outflow markers 170 and 180 located in the transition portion 124 or outflow portion 118. The outflow markers 170 and 180 can operate to assist in rotational orientation of the stent 102, as described in U.S. Application Publication No. 2021/0275299 A1, published Sep. 9, 2021, the contents of which are incorporated by reference herein in their entirety. Additionally, the outflow markers 170 and 180 can operate as a guide for determining a front or rear location the outflow markers 170 and 180 in 2D image during implantation, as described U.S. Application Publication No. 2021/0275299 A1, filed published Sep. 9, 2021, the contents of which are incorporated by reference herein in their entirety. In embodiments, the outflow marker 170 may be positioned on one of the commissure posts 126A and the outflow marker 180 may be positioned on one of the axial struts 126B.

In embodiments, the outflow markers 170 and 180 can be secured in respective containment members 172 and 182 that can be configured as a hollow structure or opening in the respective commissure posts 126A/axial struts 126B. The containment members 172, 182 may have an approximate ring shape, which can receive the outflow markers 170, 180, respectively. In any embodiment, the containment members 172, 182 can be configured in a shape that matches a shape of the outflow markers 170, 180. For example, if the outflow markers 170 and/or 180 has a circular cross-sectional shape, the respective containment member 172, 182 can define a cavity that is circular, e.g., a hollow ring.

The inflow markers 160 can be positioned towards the inflow end 106 of the stent 102 in the inflow portion 108. In an embodiment, as illustrated in FIGS. 1A-1C, the inflow markers 160 can be positioned at a node 111. In an embodiment, as illustrated in FIG. 1B, the stent 102 can include three rows of the struts 112: a first row 140 of the struts 112 formed proximate to the inflow end 106, a second row 142 of the struts 112 formed between the first row 142 and a third row 144, and the third row 144 of struts 112 formed proximate to the transition portion 124. In embodiments, the inflow markers 160 can be positioned at various locations, axially, in the inflow portion 108. The inflow markers 160 can be positioned at an intersection of the first row 140 and the second row 142. The inflow markers 160 can be secured in containment members 162. The containment members 162 can be configured similar to the containment members 172, 182.

As illustrated in FIG. 1B, the valve structure 190 includes valve leaflets 192, for example, three valve leaflets 192. The valve leaflets are attached at their commissures to the commissure posts 126A. Additionally, bottoms of the leaflets 192 can be attached to the stent 102 and/or a skirt 195 along a margin of attachment 194.

While the stent 102 has been described in detail above, those skilled in the art will recognize that the present invention is not limited to the stent 102 described above. For example, and not by way of limitation, the present invention, as described in more detail below, can be used on a stent with more or fewer inflow rows of struts and crowns, more or fewer outflow rows of struts and crowns, more or few axial struts, commissures posts with or without outflow portions, more or fewer markers (or no markers) located in different locations, and other variations known to those skilled in the art.

FIGS. 2A-2D illustrate an embodiment of tissue bumpers 200 attached to the stent 102, according to an embodiment hereof. One skilled in the art will realize that FIGS. 2A-2D illustrate one example of tissue bumpers and that components illustrated in FIGS. 2A-2D may be removed and/or additional components may be added. Additionally, while not described in complete detail with reference to FIGS. 2A-2D, the stent 102 may include any of the components described above with reference to FIGS. 1A-1C, or may be other stents.

As illustrated in FIG. 2A, which is a simplified open, flat view of an example of the stent 102, the tissue bumper 200 is positioned in areas of the stent 102 that may have a risk of leaflet contact with the stent 102. In particular, the tissue bumper 200 is positioned on struts 112 and crowns 110 of the inflow portion 108 that surround the axial struts 126B. As illustrated, the stent 102 can include three tissue bumpers 200 that are positioned on the struts 112 and crowns 110 that surround the three axial struts 126B of the stent 102. More particularly, each tissue bumper 200 is located on the distal-most row of struts 112 and crowns 110 of the inflow portion 108. In other words, each tissue bumper 200 is located on the row of struts 112 and crowns 110 of the inflow portion closest to the axial struts 126B.

In some embodiments, each tissue bumper 200 may comprise of a tissue strip 250 (which may also be referred to as a “strip of tissue” or a “strip”), as illustrated in FIG. 2D. In some embodiments, as shown in FIG. 2D, the tissue strip 250 may be rectangular, but may instead be other shapes. The tissue strip 250 may be formed of a natural or biological material such as pericardium or another membranous tissue such as intestinal submucosa. In some embodiments, the tissue strip 250 may be a low-porosity woven fabric, such as polyester, Dacron fabric, or PTFE. In some embodiments, the tissue strip 250 may be a knit or woven polyester, such as a polyester or PTFE knit. In some embodiments, the tissue strip 250 of each tissue bumper 200 may be or may include polyester velour fabrics.

As illustrated in FIG. 2D, the tissue strip 250 can be formed to various dimensions as required to form the tissue bumper 200. For example, the tissue strip 250 can be formed to a length L₂₁ and width W₂₁. In some embodiments, the wide W₂₁ may be 1-2.5 mm. In the embodiment of FIGS. 2A-2D, described in more detail below, the length L₂₁ may be 5-15 mm. One skilled in the art will realize that any example values of dimensions described herein are examples and can vary based on various factors, such as but not limited to, to manufacturing tolerances, operating conditions, the stent design, desired coverage of the tissue bumpers, and/or other factors.

In embodiments, each tissue bumper 200 can be secured to the stent 102 by wrapping or winding the tissue strip 250 around the struts 112, the crowns 110, the nodes 111, and the axial struts 126B of the stent 102. In some embodiments, ends 202 of each tissue strip 250 of the tissue bumper 200 can be secured to the struts 112 using sutures or stitches. In some embodiments, the ends 202 of the tissue strip 250 of each tissue bumper 200 can be secured to the struts 112 by knotting the tissue strip 250.

As illustrated in FIG. 2B, which is a perspective view of the exterior of the stent 102, the stent 102 can include the skirt 195. The skirt 195 can be secured to the stent 102 by sutures or stitches 196. The skirt 195 can be attached to an interior surface or an exterior surface of the stent 102. As illustrated in FIG. 2C, which is an enlarged perspective view of a portion of the interior of the stent 102, the tissue strip 250 is wrapped around the struts 112 and passed through holes 204 in the skirt 195 where the skirt 195 is attached to the struts 112. In some embodiments, the holes 204 can be cut in the skirt 195 prior to attachment to the stent 102. In some embodiments, the holes 204 can be cut in the skirt 195 at the time of attachment of the tissue bumper 200. In some embodiments, the skirt 195 may have a serrated or wavy edge that forms holes 204 between the serrated edge of the skirt 195 and the struts 112 to which the skirt 195 is attached when the skirt 195 is attached to the stent 102.

In some embodiments, the tissue strip 250 that forms each tissue bumper 200 is first secured to the axial strut 126B by forming an overlapping knot 206 that secures a central or mid-portion of the tissue strip 250 to the axial strut 126B. That is, the tissue strip 250 is wrapped generally perpendicular to the longitudinal axis of the axial strut 126B around the axial strut 126B and knotted on the exterior of stent 102 to secure the tissue strip 250. For example, as illustrated in FIG. 2C, a central or mid-portion of the tissue strip 250 can be aligned with the axial strut 126B and wrapped around the axial strut 126B such that a first end portion of the tissue strip 250 overlaps a second end portion of the tissue strip 250. While FIG. 2C illustrates a single horizontal wrapping around the axial strut 126B, the horizontal wrapping of the tissue strip 250 may extend as far up the axial strut 126B as needed and can be secured at each wrap to the axial strut 126B.

Once secured to the axial strut 126B, the tissue strip 250 is wrapped in a spiral pattern in opposite directions around each strut 112 that extends from the axial strut 126B to the node 111. During wrapping, the holes 204 operate as a guide for wrapping the tissue strip 250. For example, as illustrated, the tissue strip 250 can be wrapped around each strut 112 extending from the axial strut 126B two times, passing through two holes 204. The tissue strip 250 adjacent to the axial strut 126B is wrapped from the exterior of the stent 102 to the interior of the stent 102 such that the portion of the tissue bumper 200 at the joint (intersection) of the strut 112 and axial strut 126B is on the interior side of the stent 102 where abrasion may occur. At the node 111, the tissue strip 250 that forms the tissue bumper 200 is vertically wrapped to fully cover the node 111 where abrasion may occur. As illustrated in FIG. 2B, once the node 111 is vertically wrapped, the tissue strip 250 is wrapped in a spiral pattern (using the holes 204 as guides) along the next strut 112 until a crown 110 is reached. In the embodiment shown, without covering the crown 110, the tissue strip 250 is wrapped in a spiral pattern (using the holes 204 as guides) along the next strut 112 until the end 202 of the tissue strip 250 is reached. However, this is not meant to be limiting and the tissue strip may be wrapped around the crown 110 as well. The end 202 is then secured to the strut 112 using sutures and/or knots. This process is repeated for both directions away from the axial strut 126B.

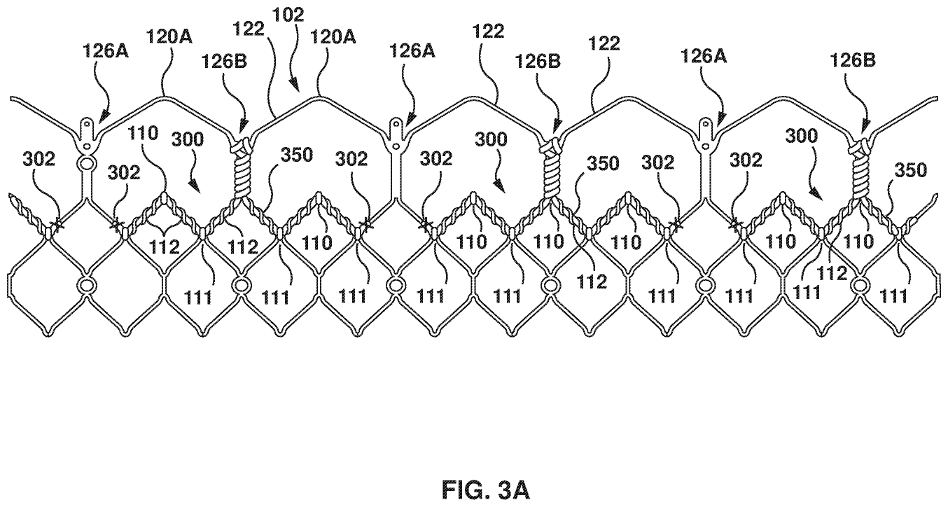

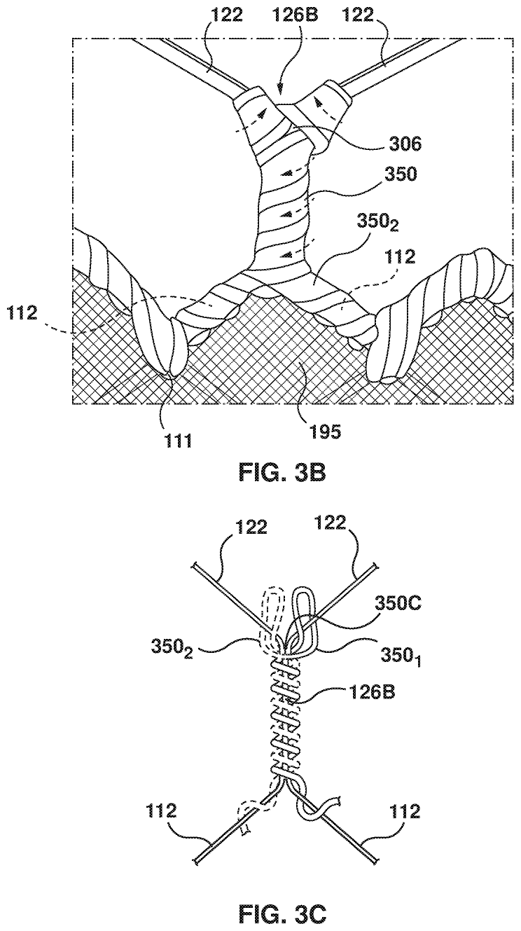

FIGS. 3A-3C illustrate tissue bumpers 300 attached to the stent 102, according to an embodiment hereof One skilled in the art will realize that FIGS. 3A-3C illustrate one example of a tissue bumper and that existing components illustrated in FIGS. 3A-3C may be removed and/or additional components may be added. Additionally, while not described in complete detail with reference to 3A-3C, the stent 102 can include any of the components described above with reference to FIGS. 1A-1C, and/or other features of stents know to those skilled in the art.

As illustrated in FIG. 3A, which is a simplified open, flat view of an example of the stent 102 with the tissue bumper 300, the tissue bumper 300 is positioned in areas of the stent 102 that may have a risk of leaflet contact with the stent. In particular, the tissue bumper 300 is positioned on the axial struts 126B and on struts 112 of the inflow portion 108 that surround the axial struts 126B. As illustrated, the stent 102 can include three tissue bumpers 300 that are positioned on the three axial struts 126B and the struts 112 that surround the three axial struts 126B of the stent 102. In this embodiment, the tissue bumper 300 can cover the entire length of the three axial struts 126B.

In embodiments, the tissue bumper 300 may comprise the tissue strip 350, as described above with respect to the tissue strip 250 in FIG. 2D. In embodiments, the tissue strip 350 that forms the tissue bumper 300 may be formed of natural materials, synthetic materials, and combinations thereof, as described above with reference to FIGS. 2A-2D. In embodiments, the tissue strip 350 that forms the tissue bumper 300 can be secured to the stent 102 by wrapping or winding the tissue strip 350 around the struts 112, the nodes 111, and the axial struts 126B. In some embodiments, ends 302 of the tissue strip 350 can be secured to the struts 112 using sutures or stitches. In some embodiments, the ends 302 of the tissue strip 350 can be secured to the struts 112 by knotting the tissue strip 250 around the struts 112.

As illustrated in FIGS. 3B and 3C, the a central portion or mid-portion 350c of the tissue strip 350 that forms each tissue bumper 300 is first secured at an intersection of the axial strut 126B with the struts 122 of the outflow portion 118 of the stent 102 by forming an overlapping knot 306 that secures the tissue strip 350 to an outflow end of the axial strut 126B and to the struts 122. That is, the tissue strip 350 is wrapped around the intersection of the axial strut 126B and the struts 122, and knotted on an exterior of the stent 102 to secure the tissue strip 350 in place during crimping of the stent 102 and subsequent deployment thereof. For example, as illustrated in FIGS. 3B and 3D, a central portion or mid-portion 350c of the tissue strip 350 can be aligned with an outflow end of the axial strut 126B. A first end portion 350₁ of the tissue strip 350 is wrapped around the intersection of the axial strut 126B and a first of the struts 122 extending from the axial strut 126B. Then, a second end portion 350₂ of the tissue strip 320 is wrapped around the intersection of the axial strut 126B and a second of the struts 122 extending from the axial strut 126B, such the second end portion of the tissue strip 350 overlaps the first end portion of the tissue strip 350. As best seen in FIG. 3C, which is a schematic view from an interior of the stent 102, the first and second end portions 350₁ and 350₂ are wrapped around their respective struts 122 and then back towards the inflow end 106 of the stent 102, passing between the central portion 350c and the axial strut 326B to form the knot 306.

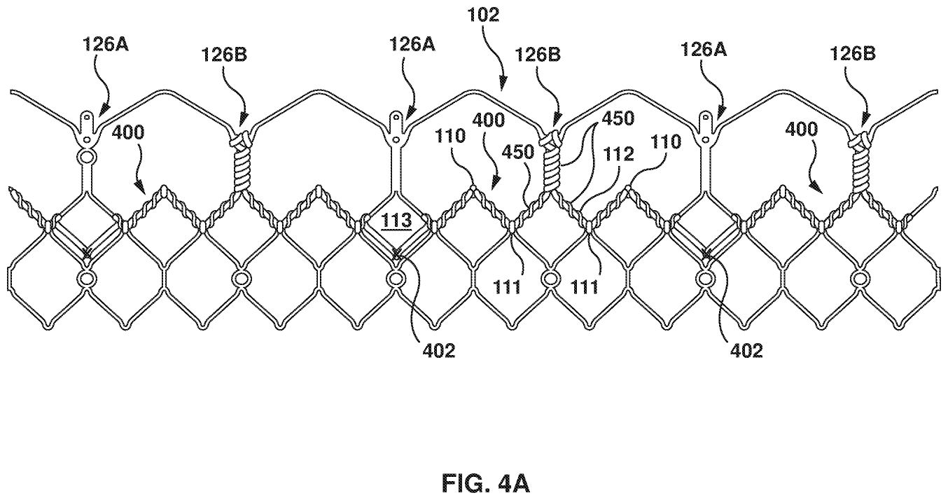

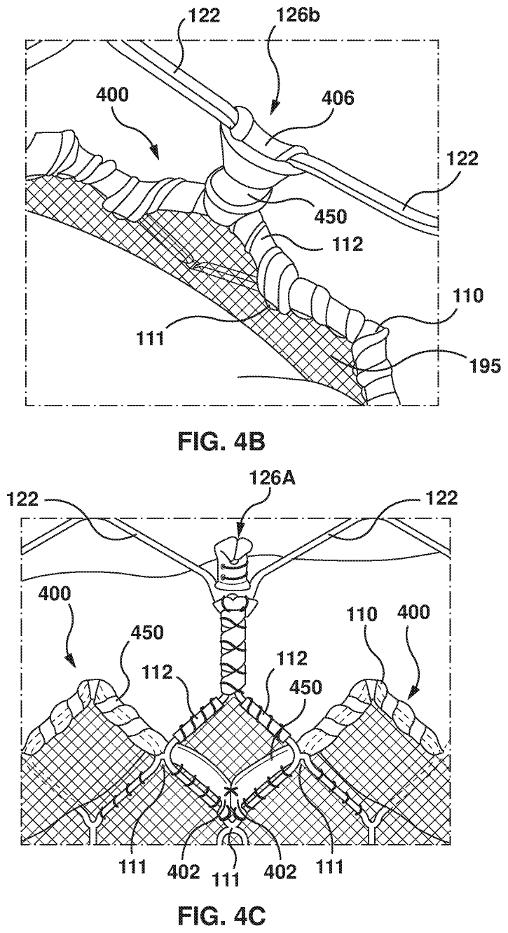

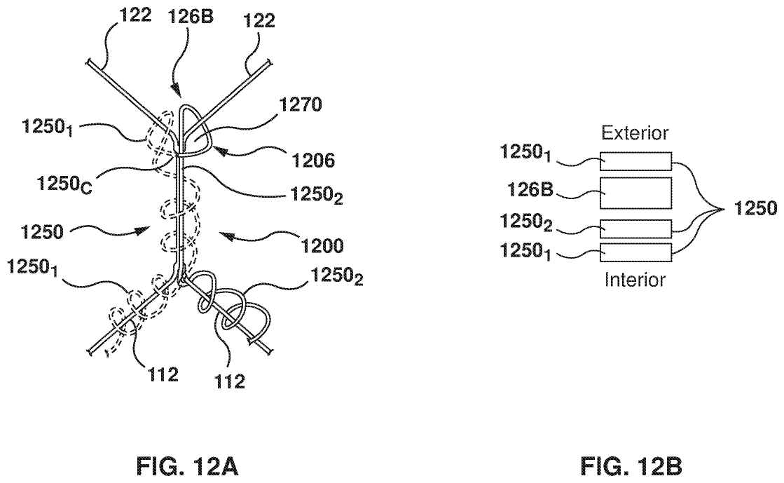

Once the knot 306 is formed, the first and second end portions 350₁, 350₂ of the tissue strip 350 can be wrapped, in the same direction, in a spiral pattern down the axial strut 126B towards the inflow end of the stent 102 such that the first and second end portions of the tissue strip 350 do not overlap (e.g., a barber pole wrap). While FIGS. 3B and 3C illustrate wrapping around the axial strut 126B in a non-overlapping pattern, the horizontal wrapping of the tissue strip 350 can be wrapped using different patterns that allow the first end portion and the second end portion of the tissue strip 350 to overlap. For example, the first end portion and the second end portion of the tissue strip 350 can be wrapped in opposite directions such that the first end portion and the second end portion of the tissue strip 350 overlap (e.g., a weave wrap). In another example, after forming the knot 306, the first end portion 350₁ of the tissue strip 350 can be positioned vertically down the length of the interior surface of the axial strut 126B, and the second end portion 350₂ of the tissue strip 350 can be wrapped around the axial strut 126B and the first end portion of the tissue strip 350, thereby securing the first end portion of the tissue strip 350 to the axial strut 126B (e.g., overlay wrap). The overlay wrap is illustrated in further detail below in FIG. 4B and FIGS. 12A-12B.

Once secured to the axial strut 126B and having been wrapped to the inflow end of the axial strut 126B, the first end portion of the tissue strip 350 is wrapped in a spiral pattern along the strut 112 extending in a first direction from the axial strut 126B to the node 111 adjacent the axial strut 126B in the first direction. Similarly, the second end portion of the tissue strip 350 is wrapped in a spiral pattern along the strut 112 extending in a second direction from the axial strut 126B to the node 111 adjacent the axial strut 126B in the second direction. As illustrated in FIG. 3C, which is an enlarged view of the interior of the stent 102, the tissue strip 350 is wrapped around the struts 112 and passed through holes 304 in the skirt 195. In some embodiments, the holes 304 can be cut in the skirt 195 prior to attachment to the stent 102. In some embodiments, the holes 304 can be cut in the skirt 195 at the time of attachment of the tissue bumper 300. In some embodiments, the skirt 195 may have a serrated or wavy edge that forms holes 304 between the serrated edge of the skirt 195 and the struts 112 to which the skirt 195 is attached when the skirt 195 is attached to the stent 102. During wrapping, the holes 304 operate as a guide for wrapping the tissue strip 350. For example, as illustrated, the tissue strip 350 can be wrapped around the strut 112 two times, passing through two holes 304. The wrapping of tissue strip 350 adjacent to the axial strut 126B is done from the exterior of the stent 102 to the interior of the stent 102 such that the tissue bumper 300 at the joint (intersection) of the strut 112 and axial strut 126B is on the interior side of the stent 102 where abrasion may occur.

As illustrated in FIG. 3C, at the nodes 111, the tissue strip 350 is vertically wrapped to fully cover the node 111 where abrasion may occur. As illustrated in FIG. 3B, once the nodes 111 are vertically wrapped, the respective end portions of the tissue strip 350 are wrapped in a spiral pattern (using the holes 304 as guides) along the next strut 112 until a crown 110 is reached. In some embodiments, without covering the crowns 110, the respective end portions of the tissue strip 350 are wrapped in a spiral pattern (using the holes 304 as guides) along the next strut 112 until each end 302 of the tissue strip 350 is reached. In other embodiments, the crowns 110 may be covered by the tissue strip 350. The ends 302 are then secured to the strut 112 using sutures and/or knots. As noted, this process is repeated for each direction away from the axial strut 126B for each tissue bumper 300. As seen in FIG. 3A, the embodiment of FIGS. 3A-3C includes three tissue bumpers 300, each extending along a respective axial strut 126B from the outflow end thereof to the inflow end thereof, along more than three struts 112, two nodes 111, and a crown 110 extending in each direction from the respective axial strut 126B.

In embodiments the tissue strip 350 may have a width in the range of 1 mm to 2.5 mm and may have a length in the range of 100 mm to 200 mm. However, this is not meant to be limiting and other dimensions may be used depending on the applications, such as, for example and not by way of limitation, the design of the stent, the size of the stent, and other factors.

FIGS. 4A-4C illustrate tissue bumpers 400 attached to the stent 102, according to an embodiment hereof One skilled in the art will realize that FIGS. 4A-4C illustrate one example of tissue bumpers and that existing components illustrated in FIGS. 4A-4C may be removed and/or additional components may be added. Additionally, while not described in complete detail with reference to 4A-4C, the stent 102 can include any of the components described above with reference to FIGS. 1A-1C, and/or other features of stents know to those skilled in the art.

As illustrated in FIG. 4A, which is a simplified open, flat view of an example of the stent 102, each tissue bumper 400 is positioned in areas of the stent 102 that may have a risk of leaflet contact with the stent 102. In particular, each tissue bumper 400 is positioned on the axial struts 126B and on struts 112 of the inflow portion 108 that surround the axial struts 126B. As illustrated, the stent 102 can include three tissue bumpers 400 that are positioned on the three axial struts 126B and the struts 112 that surround the three axial struts 126B of the stent 102. In this embodiment, the tissue bumper 400 can cover the entire length of the three axial struts 126B.

In embodiments, each tissue bumper 400 may comprise a tissue strip 450, similar to the tissue strip 250 as described above with respect to FIG. 2D. In embodiments, the tissue strip 450 may be formed of natural materials, synthetic materials, and combinations thereof, as described above with reference to FIGS. 2A-2D. In embodiments, the tissue strip 450 can be secured to the stent 102 by wrapping or winding the tissue strip 450 around the struts 112, the nodes 111, and the axial struts 126B. In some embodiments, ends 402 of the tissue strip 450 can be secured to the struts 112 using sutures or stitches. In some embodiments, the ends 402 of the tissue strip 450 can be secured to the struts 112 by knotting the tissue strip 450.

As illustrated in FIG. 4B, the tissue strip 450 is first secured to at an intersection of the axial strut 126B with struts 122 of the outflow portion 118 of the stent 102 by forming an overlapping knot 406 that secures the tissue strip 450 to the axial strut 126B and struts 122. That is, the tissue strip 450 is wrapped around the intersection of the axial strut 126B and the struts 122, and knotted on the exterior of the stent 102 to secure the tissue strip 450 in place. For example, as illustrated in FIG. 4B, a central portion or mid-portion of the tissue strip 450 can be aligned with an outflow end of the axial strut 126B. A first end portion of the tissue strip 450 is wrapped around the intersection of the axial strut 126B and a first one of the struts 122 extending from the axial strut 126B. Then, a second end portion of the tissue strip 450, opposite the first end portion with respect to the central portion, is wrapped around the intersection of the axial strut 126B and a second one of the struts 122 extending from the axial strut 126B, such the second end portion of the tissue strip 450 overlaps the first end portion of the tissue strip 450.

As illustrated in FIG. 4B, once the knot 406 is formed, the first end portion of the tissue strip 450 can be positioned vertically down the length of the interior surface of the axial strut 126B towards the inflow end of the stent 102, and the second end portion of the tissue strip 450 can be wrapped around the axial strut 126B and the first end portion of the tissue strip 450, thereby securing the first end portion of the tissue strip 450 to the axial strut 126B (e.g., an overlay wrap). While FIG. 4B illustrates wrapping around the axial strut 126B in an overlapping pattern, the horizontal wrapping of the tissue strip 450 can be wrapped using different patterns that allow the first and second end portions of the tissue strip to overlap. For example, the first and second end portions of the tissue strip 450 can be wrapped in opposite directions such that the first and second ends portions of the tissue strip 450 overlap (e.g., a weave wrap). In another example, after forming the knot 406, the first and second end portions of the tissue strip 450 can be wrapped, in the same direction, in a spiral pattern down the axial strut 126B such that the first and second end portions of the tissue strip 450 do not overlap (e.g., a barber pole wrap). The overlay wrap is further described below with respect to FIGS. 12A-12B. The barber pole wrap is illustrated in further detail above in FIGS. 3B-3C.

Once secured to the axial strut 126B, the first end portion of the tissue strip 450 is wrapped in a spiral pattern one of the struts 112 extending in a first direction from the axial strut 126B to the node 111. As illustrated in FIGS. 4B and 4C, which is an enlarged view of the exterior of the stent 102, the tissue strip 450 is wrapped around the strut 112 and passed through holes 404 in the skirt 195. In some embodiments, the holes 404 can be cut in the skirt 195 prior to attachment to the stent 102. In some embodiments, the holes 404 can be cut in the skirt 195 at the time of attachment of the tissue strip 450. In some embodiments, the skirt 195 may have a serrated or wavy edge that forms holes 404 between the serrated edge of the skirt 195 and the struts 112 to which the skirt 195 is attached when the skirt 195 is attached to the stent 102. During wrapping, the holes 404 operate as a guide for wrapping the tissue strip 450. For example, as illustrated, the tissue strip 450 can be wrapped around the strut 112 two times, passing through two holes 404. The tissue strip 450 adjacent to the axial strut 126B is wrapped from the exterior to the interior of the stent 102 such the tissue bumper 400 at the joint (intersection) of the strut 112 and axial strut 126B is disposed on the interior side of the stent 102 where abrasion may occur.

As illustrated in FIG. 4B, continuing with the first end portion of the tissue strip 450, at the first node 111 in the first direction from the axial strut 126B, the tissue strip 450 is vertically wrapped to fully cover the node 111 where abrasion may occur. Once the node 111 is vertically wrapped, the first end portion of the tissue strip 450 continues to be wrapped in a spiral pattern (using the holes 404 as guides) in the first direction along the next strut 112 until a crown 110 is reached. In the embodiment as shown in FIGS. 4B and 4C, the first end portion of the tissue strip 450 is wrapped around the crown 110 and then in a spiral pattern (using the holes 404 as guides) in the first direction along the next strut 112 until a second node 111 is reached. However, as noted in other embodiments, the crown 110 in some embodiments may not be wrapped.

When the first end portion of the tissue strip 450 reaches the second node 111 in the first direction from the axial strut 126B, the first end portion of the tissue strip 450 is wrapped from the interior of the stent 102 to an exterior of the skirt 195. The first end portion of the tissue strip 450 runs along an exterior of the skirt 195 generally parallel to the strut extending from the second node 111 to the next adjacent node 111 in the first direction and towards the inflow end of the stent 102, as shown in FIGS. 4A and 4C. The end 402 of the first end portion of the tissue strip 450 is generally aligned below (towards the inflow end) the commissure post 126A adjacent the axial strut 126B in the first direction, as shown in FIGS. 4A and 4C. The wrapping described above with respect to the first end portion of the tissue strip 450 in the first direction from the axial strut 126B is repeated for the second end portion of the tissue strip 450 in the second direction. Further, in the embodiment shown, there are three tissue bumpers 400 and thus three tissue strips 450. Therefore, an end of a second end portion of a second one of the tissue strips 450 that follows the wrapping pattern described above ends at the same location as the first end portion described above. Thus, ends 402 of two different tissue strips 450 are adjacent to each other below each of the commissure posts 126A, as best seen in FIG. 4A. These ends 402 may be secured to each other and/or to the skirt 195. If the ends 402 are secured to each other, it converts the three tissue bumpers 400 into a single or continuous tissue bumper.

In embodiments the tissue strip 450 may have a width in the range of 1 mm to 2.5 mm and may have a length in the range of 100 mm to 200 mm. However, this is not meant to be limiting and other dimensions may be used depending on the applications, such as, for example and not by way of limitation, the design of the stent, the size of the stent, and other factors.

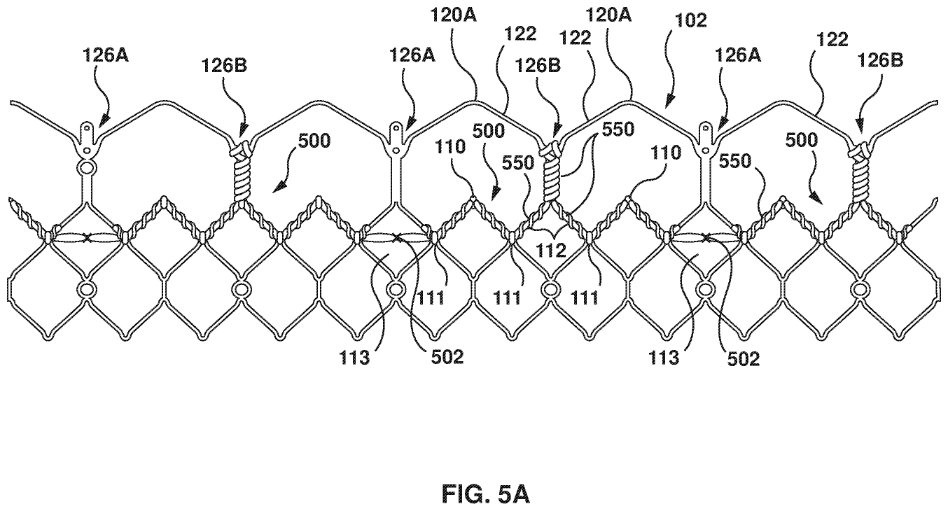

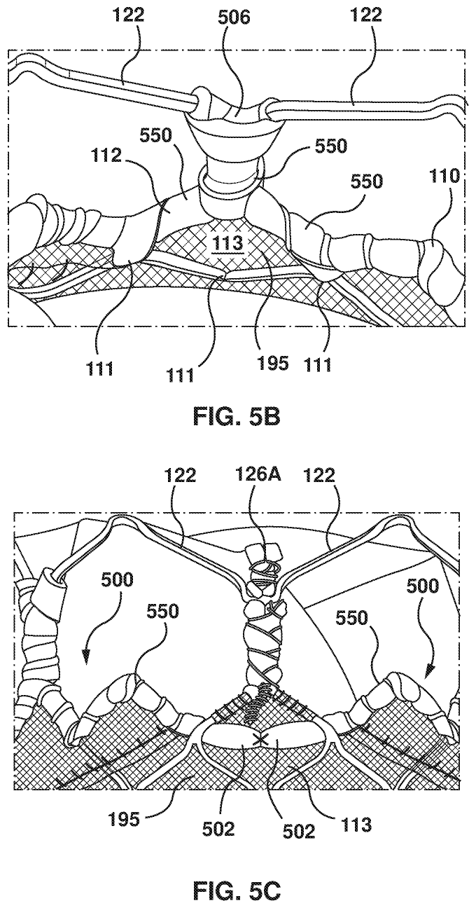

FIGS. 5A-5C illustrate tissue bumpers 500 attached to a stent, such as stent 102, according to embodiment hereof One skilled in the art will realize that FIGS. 5A-5C illustrate one example of tissue bumpers and that existing components illustrated in FIGS. 5A-5C may be removed and/or additional components may be added. Additionally, while not described in complete detail with reference to 5A-5C, the stent 102 can include any of the components described above with reference to FIGS. 1A-1C, and/or other features of stents know to those skilled in the art.

As illustrated in FIG. 5A, which is a simplified open, flat view of an example of the stent 102, the tissue bumpers 500 are positioned in areas of the stent 102 that may have a risk of leaflet contact with the stent 102. In particular, the tissue bumpers 500 are positioned on the axial struts 126B and on struts 112 of the inflow portion 108 that surround the axial struts 126B. As illustrated, the stent 102 includes three tissue bumpers 500, with each tissue bumper positioned on one of the three axial struts 126B and the struts 112 that surround the three axial struts 126B of the stent 102. In this embodiment, each tissue bumper 500 can cover the entire length of a respective one of the three axial struts 126B.

In embodiments, each tissue bumper 500 may comprise a tissue strip 550, as described above with reference to FIG. 2D. In embodiments, the tissue strip 550 may be formed of natural materials, synthetic materials, and combinations thereof, as described above with reference to FIGS. 2A-2D. In embodiments, the tissue strip 550 can be secured to the stent 102 by wrapping or winding the tissue strip 550 around the struts 112, the nodes 111, and the axial struts 126B. In some embodiments, ends 502 of the tissue strip 550 can be secured to the struts 112 using sutures or stitches. In some embodiments, the ends 502 of the tissue strip 550 can be secured to the struts 112 by knotting the tissue strip 550 of tissue.

The arrangement and attachment of one of the tissue bumpers 500 with respect to the stent 102 will now be described. It is understood that this arrangement and attachment may be used for each of the three tissue bumpers shown in FIG. 5A. However, it is also understood that other embodiments described herein may be used in conjunction with the tissue bumper 500, or fewer than three tissue bumpers may be utilized. As illustrated in FIG. 5B, a central portion or mid-portion of the tissue strip 550 of one of the tissue bumpers 500 is first secured to an intersection of the axial strut 126B with struts 122 of the outflow portion 118 of the stent 102, by forming an overlapping knot 506 that secures the tissue strip 550 to the axial strut 126B and the struts 122. That is, the tissue strip 550 is wrapped around the intersection of the axial strut 126B and the struts 122, and knotted on the exterior of the stent 102 to secure the tissue strip 550 in place. For example, as illustrated in FIG. 5B, a central portion or mid-portion of the tissue strip 550 can be aligned with an outflow end of the axial strut 126B. A first end portion of the rectangular tissue strip 550 is wrapped around the intersection of the axial strut 126B and a first one of the struts 122 extending from the axial strut 126B. Then, a second end portion of the tissue strip 550, opposite the first end portion with respect to the central portion, is wrapped around the intersection of the axial strut 126B and a second one of the struts 122 extending from the axial strut 126B, such the second end portion of the tissue strip 550 overlaps the first end portion of the tissue strip 550.

As illustrated in FIG. 5B, once the knot 506 is formed, the first end portion of the tissue strip 550 can be positioned vertically down (i.e., towards the inflow end) the length of the interior surface of the axial strut 126B, and the second end portion of the tissue strip 550 can be wrapped around the axial strut 126B and the first end portion of the tissue strip 550, thereby securing the first end portion of the tissue strip 550 to the axial strut 126B (e.g., an overlay wrap). While FIG. 5B illustrates wrapping around the axial strut 126B in an overlapping pattern, the tissue strip 550 can be wrapped using different patterns that allow the first and second end portions the tissue strip to overlap. For example, the first and second end portions of the tissue strip 550 can be wrapped in opposite directions such that the first and second end portions of the tissue strip 550 overlap (e.g., a weave wrap). In another example, after forming the knot 506, the first and second end portions of the tissue strip 550 can be wrapped, in the same direction, in a spiral pattern down the axial strut 126B such that the first and second end portions of the tissue strip 550 do not overlap (e.g., a barber pole wrap). The overlay wrap is further described below with respect to FIGS. 12A-12B. The barber pole wrap illustrated in further detail in FIGS. 3B-3C.

Once secured to the axial strut 126B, the first end portion the tissue strip 550 is wrapped in a spiral pattern in a first direction along one of the struts 112 extending in the first direction from the axial strut 126B to the first node 111 adjacent the axial strut 126B in the first direction. As illustrated in FIGS. 5B and 5C, the tissue strip 550 is wrapped around the struts 112 and passed through holes 504 in the skirt 195. In some embodiments, the holes 504 can be cut in the skirt 195 prior to attachment to the stent 102. In some embodiments, the holes 504 can be cut in the skirt 195 at the time of attachment of the tissue bumper 500. In some embodiments, the skirt 195 may have a serrated or wavy edge that forms holes 504 between the serrated edge of the skirt 195 and the struts 112 to which the skirt 195 is attached when the skirt 195 is attached to the stent 102. During wrapping, the holes 504 operate as a guide for wrapping the tissue strip 550. For example, as illustrated, the tissue strip 550 can be wrapped around the strut 112 two times, passing through two holes 504, before reaching the first node 111. In some embodiments, adjacent to the inflow end of the axial strut 126B, the tissue strip 550 is wrapped from the exterior to the interior such that the tissue bumper 500 at the joint (intersection) of the strut 112 and axial strut 126B is on the interior side of the stent 102 where abrasion may occur.

As illustrated in FIG. 5C, at the first node 111 in the first direction, the first end portion of the tissue strip 550 is vertically wrapped to fully cover the first node 111 where abrasion may occur. Once the first node 111 is vertically wrapped, the first end portion of the tissue strip 550 continues to be wrapped in a spiral pattern (using the holes 504 as guides) along the next strut 112 in the first direction until a crown 110 is reached. The tissue strip 550 is wrapped around the crown 110, and continues to be wrapped in a spiral pattern (using the holes 504 as guides) along the next strut 112 in the first direction until a second node 111 in the first direct is reached. As noted, in some embodiments, the crowns 110 are not wrapped with the tissue strip 550.

In the embodiment of FIGS. 5A-5C, when the first end portion of the tissue strip 550 reaches the second node 111 in the first direction, the tissue strip 550 is wrapped from the interior of the skirt 195 to the exterior of the skirt 195, and extends along the exterior of the skirt 195 in a circumferential direction. In other words, the tissue strip 550 extends across the side opening 113 from the second node 111 to a first node 111 in the first direct adjacent the commissure post 126A, as best seen in FIGS. 5A and 5C. The tissue strip 550 is of a length such that the end 502 of the first end portion of the tissue strip is axially aligned with the commissure post 126A, i.e., the end 502 is aligned and towards the inflow end of the stent 102. Due to the arrangement of the stent 102, the end 502 is also generally in the center of the side opening 113.

This process is repeated in a second direction opposite the first direction with respect to the axial strut 126B with the second end portion of the tissue strip 550 to complete one of the tissue bumpers 500. As noted above, the embodiment shown includes three tissue bumpers 500. Therefore, there is a second tissue bumper 500 with a tissue strip 550 that extends in the second direction from a second one of the of the axial struts 126B towards the commissure post 126A below which the first end 502 of the tissue strip of the first tissue bumper 500 resides. These ends 502 of two different tissue bumpers 500 disposed adjacent to each other can be seen in FIGS. 5A and 5C. The adjacent ends 502 can be secured to the skirt 195 and to each other, such as by stitching. If the adjacent ends 502 are attached to each other, this makes the three tissue bumpers 500 into a single or continuous tissue bumper, as best seen in FIG. 5A.

In embodiments the tissue strip 550 may have a width in the range of 1 mm to 2.5 mm and may have a length in the range of 100 mm to 200 mm. However, this is not meant to be limiting and other dimensions may be used depending on the applications, such as, for example and not by way of limitation, the design of the stent, the size of the stent, and other factors.

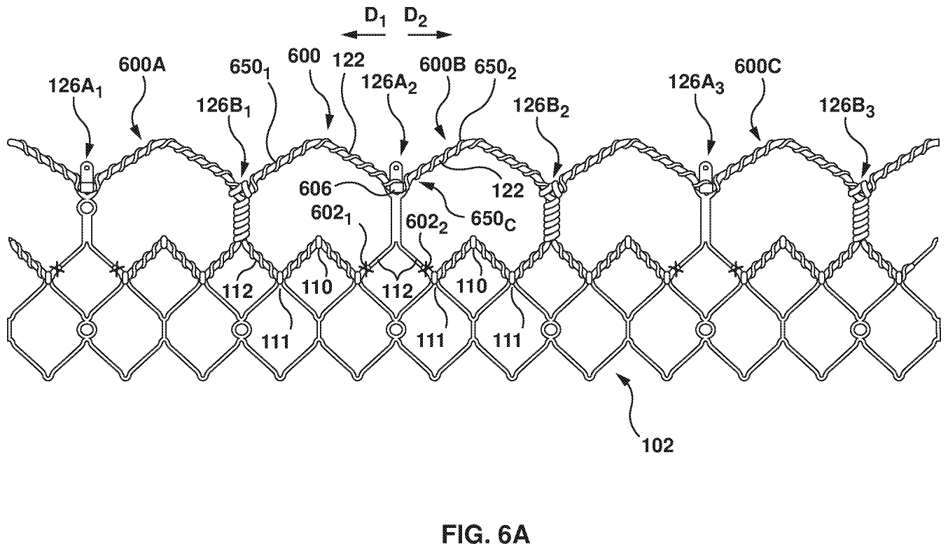

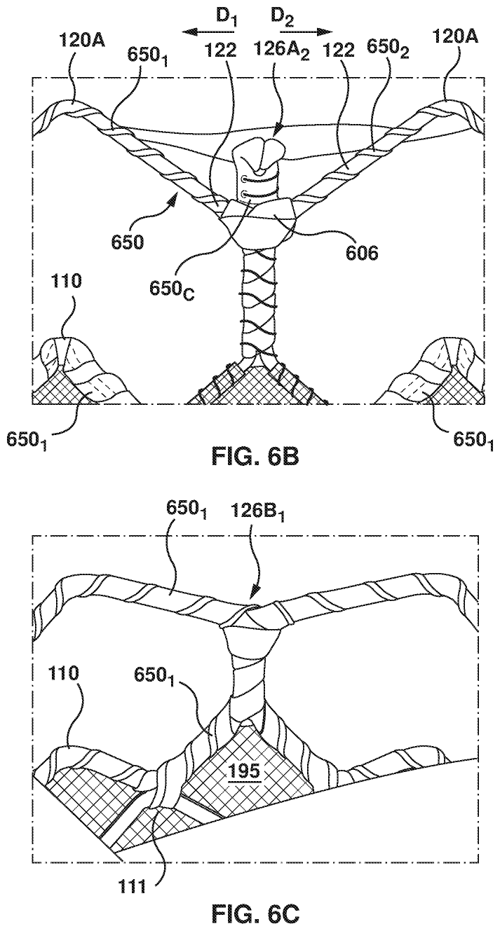

FIGS. 6A-6C illustrate an embodiment including three tissue bumpers 600 attached to a stent, such as the stent 102. One skilled in the art will realize that FIGS. 6A-6C illustrate one example of tissue bumpers and that existing components illustrated in FIGS. 6A-6C may be removed and/or additional components may be added. Additionally, while not described in complete detail with reference to 6A-6C, the stent 102 can include any of the components described above with reference to FIGS. 1A-1C, and/or other features of stents know to those skilled in the art.

As illustrated in FIG. 6A, which is a simplified open, flat view of an example of the stent 102, the tissue bumpers 600 are positioned in areas of the stent 102 that may have a risk of leaflet contact with the stent 102. In particular, the tissue bumpers 600 are positioned on the struts 122 of the outflow portion 118, the axial struts 126B, and the struts 112 of the inflow portion 108 that surround the axial struts 126B. As illustrated, the stent 102 can include three tissue bumpers 600 that are each positioned on the two of three axial struts 126B, and the struts 112 and 122 that surround the three axial struts 126B of the stent 102. The three tissue bumpers 600 will be referred to herein individually as the tissue bumper 600A, 600B, and 600C when describing an individual tissue bumper for clarity. for In this embodiment, the tissue bumpers 600 can cover the entirety of the axial struts 126B, the struts 112 at the outflow end of the inflow portion 108, and the struts 122 that form the outflow portion 118.

In embodiments, each tissue bumper 600 may comprise a tissue strip 650, such as the tissue strip 250 described above with respect to FIG. 2D. In embodiments, the tissue strip 650 may be formed of natural materials, synthetic materials, and combinations thereof, as described above with reference to FIGS. 2A-2D. In embodiments, the rectangular tissue strip 650 can be secured to the stent 102 by wrapping or winding the tissue strip 650 around the commissure posts 126A, the axial struts 126B, the struts 122, the struts 112, and the nodes 111. In some embodiments, ends 602 of the tissue strip 650 can be secured to the struts 112 using sutures or stitches. In some embodiments, the ends 602 of the tissue strip 650 can be secured to the struts 112 by knotting the tissue strip 650.

The arrangement and attachment of one of the tissue bumpers 600 with respect to the stent 102 will now be described. It is understood that this arrangement and attachment may be used for each of the three tissue bumpers 600 shown in FIG. 6A. However, it is also understood that other embodiments described herein may be used in conjunction with the tissue bumper 600, or fewer than three tissue bumpers may be utilized. The tissue bumper 600B will be described herein, but as illustrated, the wrapping pattern/arrangement is the same for each of the three tissue bumpers 600, just that their locations are different. As illustrated in FIG. 6B, which is a perspective view of the exterior of the stent 102, a central of mid-portion 650c of the tissue strip 650 forming the tissue bumper 600B is first secured to a commissure post 126A₂ at an intersection of the commissure post 126A₂ and the two struts 122 extending from the commissure post 126A₂ by forming an overlap knot 606 that secures the tissue strip 650 to the commissure post 126A₂. That is, the tissue strip 650 is wrapped around the commissure post 126A₂ at the intersection of the commissure post 126A₂ and the two struts 122, and knotted on exterior of stent 102 to secure the tissue strip 650 in place. For example, as illustrated in FIG. 6B, the central portion of mid-portion 650c of the tissue strip 650 can be aligned with an outflow end the commissure post 126A₂ at the intersection of the commissure post 126A₂ and the two struts 122. A first end portion 650₁ and a second end portion 650₂ of the rectangular tissue strip 650, disposed on opposite sides of the central portion 650c, are wrapped around the commissure post 126A₂ to form the overlap knot 606.

Once the knot 606 is formed, the first end portion 650₁ of the tissue strip 650 is wrapped spirally around the struts 122 extending from the commissure post 126A₂ in a first direction D₁ (left in FIGS. 6A and 6B), and the second end portion 650₂ of the tissue strip 650 is wrapped spirally around struts 122 extending from the commissure post 126A₂ in a second direction (right in FIGS. 6A and 6B), opposite the first direction D₁. The first end portion 650₁ and the second end portion 650₂ are spirally wrapped around their respective struts 122 until the first end portion 350₁ reaches axial strut 126B1 and the second end portion 650₂ reaches the axial strut 126B₂. In other words, the first end portion 650₁ is wrapped spirally around two struts 122 extending in the first direction D₁ (left in FIGS. 6A, 6B) until the adjacent axial strut 126B in the first direction D₁ is reached (axial strut 126B₁). Similarly, the second end portion 650₂ is wrapped spirally around two struts 122 extending in the second direction D₂ (right in FIGS. 6A, 6B) until the adjacent axial strut 126B in the second direction D₂ is reached (axial strut 126B₂). The first end portion 650₁ and the second end portion 650₂ of the tissue strip 650 are then spirally wrapped around and down (towards the inflow end) the respective axial struts 126B₁, 126B₂ until the struts 112 extending from the respective inflow ends of the axial struts 126B₁, 126B₂ are reached, as illustrated in FIG. 6C, which is an enlarged view of an interior of the stent 102 at the axial strut 126B₁.

Once the struts 112 are reached, the first end portion 650₁ and the second end portion 650₂ of the tissue strip 650 are spirally wrapped around their respective struts 112 in a direction back towards the commissure post 126A to which the central or mid-portion 650c of the tissue strip 650 was secured, in this example the commissure post 126A₂. Thus, the first end portion 650₁ of the tissue strip 650 is wrapped in a spiral pattern in the second direction D₂ (back towards the commissure post 126A₂, to the right in FIG. 6A) along the strut 112 from the axial strut 126B₁ to the first node 111 adjacent to the axial strut 126B in the second direction. As illustrated in FIG. 6C, the first end portion 650₁ of the tissue strip 650 is wrapped around the struts 112 and passed through holes 604 in the skirt 195. It is noted that the directions D₁ and D₂ would be reversed in FIG. 6C because the view is from the inside of the stent 102. In some embodiments, the holes 604 can be cut in the skirt 195 prior to attachment to the stent 102. In some embodiments, the holes 604 can be cut in the skirt 195 at the time of attachment of the tissue bumper 600. In some embodiments, the skirt 195 may have a serrated or wavy edge that forms holes 604 between the serrated edge of the skirt 195 and the struts 112 to which the skirt 195 is attached when the skirt 195 is attached to the stent 102. During wrapping, the holes 604 operate as a guide for wrapping the first end portion 650₁ and the second end portion 650₂ of the tissue strip 650 that forms the tissue bumper 600. For example, as illustrated, the first end portion 650₁ of the tissue strip 650 can be wrapped around the first strut 112 two times, passing through two holes 604. The first end portion 650₁ of the tissue strip 650 at the junction of the axial strut 126B₁ and the strut 112 extending therefrom is wrapped from the exterior of the stent 102 to the interior of the stent 102 such that tissue bumper 600 at the joint (intersection) of the strut 112 and axial strut 126B₁ is on the interior side of the stent 102 where abrasion may occur.

As illustrated in FIG. 6C, at the first node 111, the first end portion 650₁ of the tissue strip 650 is vertically wrapped to fully cover the first node 111 where abrasion may occur. Once the first node 111 is vertically wrapped, the first end 650₁ of the tissue strip 650 continues to be wrapped in the second direction D₂ in a spiral pattern (using the holes 604 as guides) along the next strut 112 at the outflow end of the inflow portion 108 until a crown 110 is reached. The first end portion 650₁ of the tissue strip 650 is wrapped in a spiral pattern past the crown 110 (either covering or not covering the crown 110) and along the next strut 112 (using the holes 604 as guides) until the end 602₁ of the tissue strip 650. The end 602₁ is then secured to the strut 112 using sutures and/or knots. As can be seen in FIG. 6A, the end 602₁ of the first end portion 650₁ of the tissue strip 650 is on the strut 112 that extends in the first direction D₁ from the inflow end of the commissure post 126A₂ that the central or mid-portion 650c of the tissue strip 650 is attached to at the outflow end. This wrapping process/pattern is repeated for the second end portion 650₂ of the tissue strip 650 after it is wrapped around and to the inflow end of the axial strut 126B₂, such that the second portion 650₂ is wrapped around the struts 112, crowns 110, and nodes 111 at the outflow end of the inflow portion in the first direction D₁ back towards the commissure post 126A₂.

In this embodiment, as illustrated in FIGS. 6A and 6C, the first end portion 650₁ of a tissue strip 650 and the second end portion 650₂ of an adjacent rectangular tissue strip 650 are wrapped around the same axial strut 126B. Thus, for example, as shown in FIG. 6A, at the axial strut 126B₁, the first end portion 650₁ of the tissue strip 650 of the tissue bumper 600B and the second end portion of the tissue strip 650 of the tissue bumper 600A are both wrapped down (towards the inflow end) of the axial strut 126B₁. This pattern is repeated for the other axial struts 126B and tissue bumpers 600. In some embodiments, the first end portion 650₁ and the second end portion 650₂ of adjacent tissue strips 650 can be wrapped, in the same direction, in a spiral pattern down the axial strut 126B such the first end portion 650₁ and the second end portion 650₂ of the adjacent rectangular tissue strips 650 do not overlap (e.g., a barber pole wrap). In other embodiments, the tissue strips 650 can be wrapped using different patterns that allow the first end portion 650₁ and the second end portion 650₂ of the adjacent tissue strips 650 to overlap. For example, the first end portion 650₁ and the second end portion 650₂ of the adjacent tissue strips 650 can be wrapped in opposite directions such that the first end portion 650₁ and the second end portion 650₂ of the adjacent tissue strips 650 overlap (e.g., a weave wrap). In another example, one of the first end portion 650₁ or the second end portion 650₂ of the adjacent tissue strips 650 can be positioned vertically down the length of the interior surface of the axial strut 126B, and the other of the first end portion 650₁ or the second end portion 650₂ of the adjacent tissue strip 650 can be wrapped around the axial strut 126B, thereby securing the first end portion 650₁ or the second end portion 650₂ of the adjacent tissue strips 650 to the axial strut 126B (e.g., overlay wrap). The overlay wrap is further described below with respect to FIGS. 12A-12B.

In embodiments the tissue strip 650 may have a width in the range of 1 mm to 2.5 mm and may have a length in the range of 100 mm to 200 mm. However, this is not meant to be limiting and other dimensions may be used depending on the applications, such as, for example and not by way of limitation, the design of the stent, the size of the stent, and other factors.

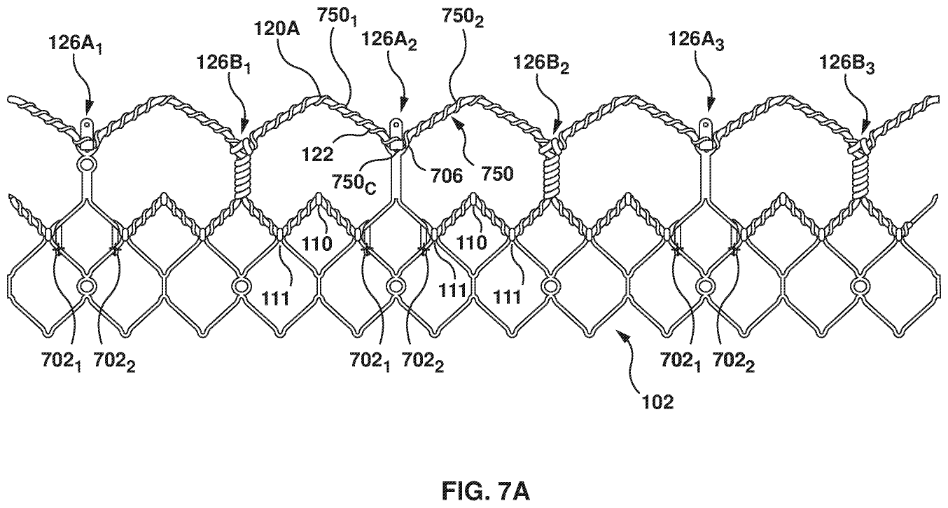

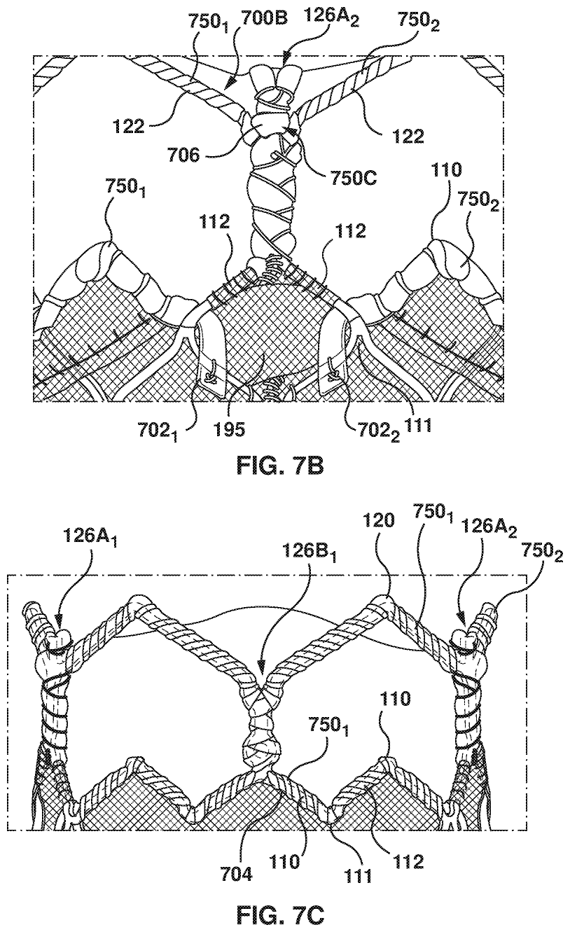

FIGS. 7A-7C illustrate an embodiment of tissue bumpers 700 attached to a stent, such as the stent 102. One skilled in the art will realize that FIGS. 7A-7C illustrate one example of tissue bumpers and that existing components illustrated in FIGS. 7A-7C may be removed and/or additional components may be added. Additionally, while not described in complete detail with reference to 7A-7C, the stent 102 can include any of the components described above with reference to FIGS. 1A-IC, and/or other features of stents known to those skilled in the art.

As illustrated in FIG. 7A, which is a simplified open, flat view of an example of the stent 102, the tissue bumpers 700 are positioned in areas of the stent 102 that may have a risk of leaflet contact with the stent 102. In particular, the tissue bumpers 700 are positioned on the struts 122 of the outflow portion 118, the axial struts 126B, and the struts 112 of the inflow portion 108 that surround the axial struts 126B. As illustrated, the stent 102 can include three tissue bumpers 700 that are each positioned on the two of three axial struts 126B, and the struts 112 and 122 that surround the three axial struts 126B of the stent 102. The three tissue bumpers 700 will be referred to herein individually as the tissue bumper 700A, 700B, and 700C when describing an individual tissue bumper for clarity. for In this embodiment, the tissue bumpers 700 can cover the entirety of the axial struts 126B, the struts 112 at the outflow end of the inflow portion 108, and the struts 122 that form the outflow portion 118.

In embodiments, each tissue bumper 700 may comprise tissue strip 750, such as the tissue strip 250 described above with respect to FIG. 2D. In embodiments, the tissue strip 750 may be formed of natural materials, synthetic materials, and combinations thereof, as described above with reference to FIGS. 2A-2D. In embodiments, the tissue strip 750 can be secured to the stent 102 by wrapping or winding the tissue strip 750 around the commissure posts 126A, the axial struts 126B, the struts 122, the struts 112, and the nodes 111. In some embodiments, ends 702 of the tissue strip 750 can be secured to the struts 112 using sutures or stitches. In some embodiments, the ends 702 of the tissue strip 750 can be secured to the struts 112 by knotting the tissue strip 750.

The arrangement and attachment of one of the tissue bumpers 700 with respect to the stent 102 will now be described. It is understood that this arrangement and attachment may be used for each of the three tissue bumpers 700 shown in FIG. 7A. However, it is also understood that other embodiments described herein may be used in conjunction with the tissue bumper 700, or fewer than three tissue bumpers may be utilized. The tissue bumper 700B will be described herein, but as illustrated, the wrapping pattern/arrangement is the same for each of the three tissue bumpers 700, just that their locations are different. As illustrated in FIG. 7B, which is a perspective view of the exterior of the stent 102, a central of mid-portion 750c of the tissue strip 750 forming the tissue bumper 700B is first secured to a commissure post 126A₂ at an intersection of the commissure post 126A₂ and the two struts 122 extending from the commissure post 126A₂ by forming an overlap knot 706 that secures the tissue strip 750 to the commissure post 126A₂. That is, the tissue strip 750 is wrapped around the commissure post 126A₂ at the intersection of the commissure post 126A₂ and the two struts 122, and knotted on the exterior of the stent 102 to secure the tissue strip 750 in place. For example, as illustrated in FIG. 7B, the central portion of mid-portion 750c of the tissue strip 750 can be aligned with an outflow end the commissure post 126A₂ at the intersection of the commissure post 126A₂ and the two struts 122. A first end portion 750₁ and a second end portion 750₂ of the tissue strip 750, disposed on opposite sides of the central portion 750c, are wrapped around the commissure post 126A₂ to form the overlap knot 706.

Once the knot 706 is formed, the first end portion 750₁ of the tissue strip 750 is wrapped spirally around the struts 122 extending from the commissure post 126A₂ in a first direction D₁ (left in FIGS. 7A and 7B), and the second end portion 750₂ of the tissue strip 750 is wrapped spirally around struts 122 extending from the commissure post 126A₂ in a second direction D₂ (right in FIGS. 7A and 7B), opposite the first direction D₁. The first end portion 750₁ and the second end portion 750₂ are spirally wrapped around their respective struts 122 until the first end portion 750₁ reaches the axial strut 126B₁ and the second end portion 750₂ reaches the axial strut 126B₂. In other words, the first end portion 750₁ is wrapped spirally around two struts 122 extending in the first direction D₁ (left in FIGS. 7A, 7B) until the adjacent axial strut 126B in the first direction D₁ is reached (axial strut 126B₁). Similarly, the second end portion 750₂ is wrapped spirally around two struts 122 extending in the second direction D₂ (right in FIGS. 7A, 7B) until the adjacent axial strut 126B in the second direction D₂ is reached (axial strut 126B₂). The first end portion 750₁ and the second end portion 750₂ of the tissue strip 750 are then spirally wrapped around and down (towards the inflow end) the respective axial struts 126B₁, 126B₂ until the struts 112 extending from the respective inflow ends of the axial struts 126B₁, 126B₂ are reached, as illustrated in FIGS. 7A and 7C, which is an enlarged view of an interior of the stent 102 at the axial strut 126B₁.

Once the struts 112 are reached, the first end portion 750₁ and the second end portion 750₂ of the tissue strip 750 are spirally wrapped around their respective struts 112 in a direction back towards the commissure post 126A to which the central or mid-portion 750c of the tissue strip 750 was secured, in this example the commissure post 126A₂. Thus, the first end portion 750₁ of the tissue strip 750 is wrapped in a spiral pattern in the second direction D₂ (back towards the commissure post 126A₂, to the right in FIG. 7A) along the strut 112 from the axial strut 126B₁ to the first node 111 adjacent to the axial strut 126B₁ in the second direction D₂. As illustrated in FIG. 7C, the first end portion 750₁ of the tissue strip 750 is wrapped around the struts 112 and passed through holes 704 in the skirt 195. In some embodiments, the holes 704 can be cut in the skirt 195 prior to attachment to the stent 102. In some embodiments, the holes 704 can be cut in the skirt 195 at the time of attachment of the tissue bumper 700. In some embodiments, the skirt 195 may have a serrated or wavy edge that forms holes 704 between the serrated edge of the skirt 195 and the struts 112 to which the skirt 195 is attached when the skirt 195 is attached to the stent 102. During wrapping, the holes 704 operate as a guide for wrapping the first end portion 750₁ and the second end portion 750₂ of the tissue strip 750. For example, as illustrated, the first end portion 750₁ of the tissue strip 750 can be wrapped around the first strut 112 two times, passing through two holes 704. The first end portion 750₁ of the tissue strip 750 at the junction of the axial strut 126B₁ and the strut 112 extending therefrom is wrapped from the exterior of the stent 102 to the interior of the stent 102 such that the tissue bumper 700 at the joint (intersection) of the strut 112 and axial strut 126B₁ is on the interior side of the stent 102 where abrasion may occur.

As illustrated in FIG. 7C, at the first node 111, the first end portion 750₁ of the tissue strip 750 is vertically wrapped to fully cover the first node 111 where abrasion may occur. Once the first node 111 is vertically wrapped, the first end 750₁ of the tissue strip 750 continues to be wrapped in the second direction D₂ in a spiral pattern (using the holes 704 as guides) along the next strut 112 at the outflow end of the inflow portion 108 until a crown 110 is reached. The first end portion 750₁ of the tissue strip 750 is wrapped in a spiral pattern past the crown 110 (either covering or not covering the crown 110) and along the next strut 112 (using the holes 704 as guides) until the next node 111 is reached. The node 111 in this embodiment is adjacent to commissure post 126A₂ where the wrapping started. In particular the node 111 is adjacent to commissure post 126A₂ in the first direction D₁. The second end portion 750₂ is similarly wrapped in the first direction D₁ from the inflow end of the axial strut 126B₂ around the struts 112, nodes 111, and crowns 110 until it reaches the node 111 adjacent the commissure post 126A₂ in the second direction.

In this embodiment, shown in FIGS. 7A and 7B, when the first end portion 750₁ and the second end portion 750₂ of the tissue strip 750 reach the respective nodes 111 on either side of the commissure post 126A₂, the respective end portion 750₁ and 750₂ are wrapped from an exterior of the stent 102 to an interior of the stent 102 over the node 111 towards the outflow end of the stent 102, then under the strut 112 (i.e., from the interior to the exterior of the stent) extending from the inflow end of the commissure post 126A₂, then generally axially or longitudinally towards the inflow end of the stent 102 on an exterior side of the skirt 195, and over the exterior of the strut 112 longitudinally adjacent to the strut 112 adjacent the commissure post 126A₂. The respective ends 702₁ and 702₂ of the tissue strip 750 are then secured to the skirt 195 using sutures and/or knots. Thus, the ends of 702₁ and 702₂ of the tissue strip 750 extend generally axially, as shown in FIGS. 7A and 7B.

Also in this embodiment, as illustrated in FIGS. 7A and 7C, the first end portion 750₁ of a tissue strip 750 and the second end portion 750₂ of an adjacent tissue strip 750 are wrapped around the same axial strut 126B. Thus, for example, as shown in FIG. 7A, at the axial strut 126B₁, the first end portion 750₁ of the tissue strip 750 of the tissue bumper 700B and the second end portion 750₂ of the tissue strip 750 of the tissue bumper 700A are both wrapped down (towards the inflow end) of the axial strut 126B₁. This pattern is repeated for the other axial struts 126B and the other tissue bumpers 700. In some embodiments, the first end portion 750₁ and the second end portion 750₂ of adjacent tissue strips 750 can be wrapped, in the same direction, in a spiral pattern down the axial strut 126B such the first end portion 750₁ and the second end portion 750₂ of the adjacent tissue strips 750 do not overlap (e.g., a barber pole wrap). The barber pole wrap is described in further detail above with respect to FIGS. 3B-3C. While FIG. 7C illustrates wrapping around the axial strut 126B₁ in a non-overlapping pattern, the tissue strips 750 can be wrapped using different patterns that allow the first end portion 750₁ and the second end portion 750₂ of the adjacent tissue strips 750 to overlap. For example, the first end portion 750₁ and the second end portion 750₂ of the adjacent tissue strips 750 can be wrapped in opposite directions such that the first end portion 750₁ and the second end portion 750₂ of the adjacent tissue strips 750 overlap (e.g., a weave wrap). In another example, one of the first end portion 750₁ or the second end portion 750₂ of the adjacent tissue strips 750 can be positioned vertically down the length of the interior surface of the axial strut 126B, and the other of the first end portion 750₁ or the second end portion 750₂ of the adjacent tissue strips 750 can be wrapped around the axial strut 126B, thereby securing the first end portion 750₁ or the second end portion 750₂ of the adjacent tissue strips 750 to the axial strut 126B (e.g., overlay wrap). The overlay wrap is further described below with respect to FIGS. 12A-12B.

As can be seen the tissue bumpers 700 of FIGS. 7A-7B are the same as the tissue bumpers 600 of FIGS. 6A-6C except at the ends of the tissue strips 750 as compared to the ends of the tissue strip 650.

In embodiments the tissue strip 750 may have a width in the range of 1 mm to 2.5 mm and may have a length in the range of 100 mm to 200 mm. However, this is not meant to be limiting and other dimensions may be used depending on the applications, such as, for example and not by way of limitation, the design of the stent, the size of the stent, and other factors.

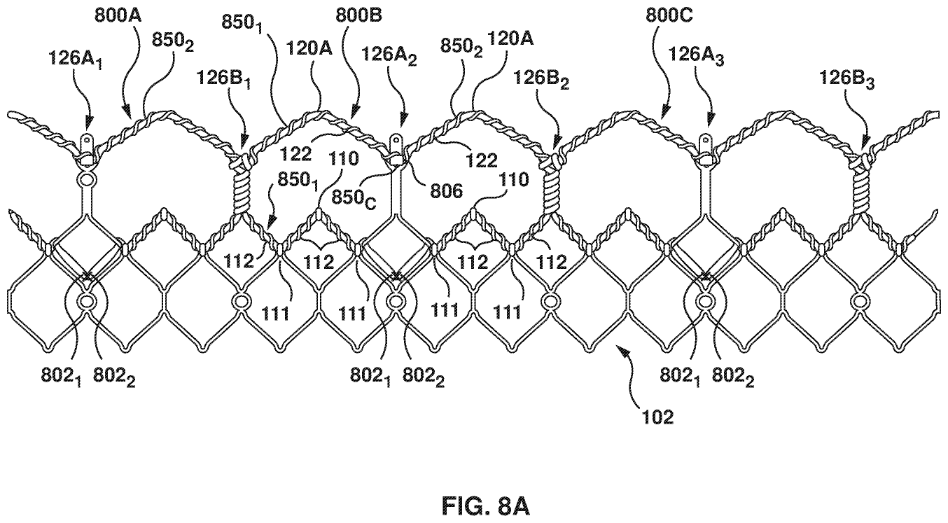

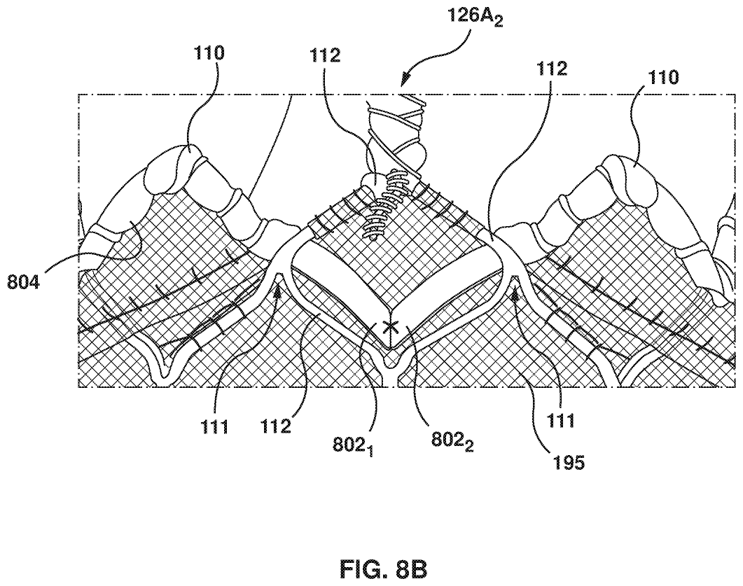

FIGS. 8A-8B illustrate an embodiment of tissue bumpers 800 attached to a stent, such as the stent 102. One skilled in the art will realize that FIGS. 8A-8B illustrate one example of tissue bumpers and that existing components illustrated in FIGS. 8A-8B may be removed and/or additional components may be added. Additionally, while not described in complete detail with reference to 8A-8B, the stent 102 can include any of the components described above with reference to FIGS. 1A-1C, and/or other features of stents known to those skilled in the art.

As illustrated in FIG. 8A, which is a simplified open, flat view of an example of the stent 102, the tissue bumpers 800 are positioned in areas of the stent 102 that may have a risk of leaflet contact with the stent 102. In particular, the tissue bumpers 800 are positioned on the struts 122 of the outflow portion 118, the axial struts 126B, and the struts 112 of the inflow portion 108 that surround the axial struts 126B. As illustrated, the stent 102 can include three tissue bumpers 800 that are each positioned on the two of three axial struts 126B, and the struts 112 and 122 that surround the three axial struts 126B of the stent 102. The three tissue bumpers 800 will be referred to herein individually as the tissue bumper 800A, 800B, and 800C when describing an individual tissue bumper for clarity. for In this embodiment, the tissue bumpers 800 can cover the entirety of the axial struts 126B, the struts 112 at the outflow end of the inflow portion 108, and the struts 122 that form the outflow portion 118.

In embodiments, each tissue bumper 800 may comprise a tissue strip 850, such as the tissue strip 250 described above with respect to FIG. 2D. In embodiments, the tissue strip 850 may be formed of natural materials, synthetic materials, and combinations thereof, as described above with reference to FIGS. 2A-2D. In embodiments, the tissue strip 850 can be secured to the stent 102 by wrapping or winding the tissue strip 850 around the commissure posts 126A, the axial struts 126B, the struts 122, the struts 112, and the nodes 111. In some embodiments, ends 802 of the tissue strip 850 can be secured to the struts 112 using sutures or stitches. In some embodiments, the ends 802 of the tissue strip 850 can be secured to the struts 112 by knotting the tissue strip 850.

The arrangement and attachment of one of the tissue bumpers 800 with respect to the stent 102 will now be described. It is understood that this arrangement and attachment may be used for each of the three tissue bumpers 800 shown in FIG. 8A. However, it is also understood that other embodiments described herein may be used in conjunction with the tissue bumper 800, or fewer than three tissue bumpers may be utilized. The tissue bumper 800B will be described herein, but as illustrated, the wrapping pattern/arrangement is the same for each of the three tissue bumpers 800, just that their locations are different. As illustrated in FIG. 8A, a central of mid-portion 850c of the tissue strip 850 forming the tissue bumper 800B is first secured to a commissure post 126A₂ at an intersection of the commissure post 126A₂ and the two struts 122 extending from the commissure post 126A₂ by forming an overlap knot 806 that secures the tissue strip 850 to the commissure post 126A₂. That is, the tissue strip 850 is wrapped around the commissure post 126A₂ at the intersection of the commissure post 126A₂ and the two struts 122, and knotted on the exterior of the stent 102 to secure the tissue strip 850 in place. For example, as illustrated in FIG. 8A, the central portion of mid-portion 850c of the tissue strip 850 can be aligned with an outflow end the commissure post 126A₂ at the intersection of the commissure post 126A₂ and the two struts 122. A first end portion 850₁ and a second end portion 850₂ of the tissue strip 850, disposed on opposite sides of the central portion 850c, are wrapped around the commissure post 126A₂ to form the overlap knot 806. A closer view detail of this wrapping can be seen in FIGS. 6B and 7B.

Once the knot 806 is formed, the first end portion 850₁ of the tissue strip 850 is wrapped spirally around the struts 122 and crowns 120A extending from the commissure post 126A₂ in a first direction D₁ (left in FIG. 8A), and the second end portion 850₂ of the tissue strip 850 is wrapped spirally around struts 122 extending from the commissure post 126A₂ in a second direction D₂ (right in FIG. 8A), opposite the first direction D₁. The first end portion 850₁ and the second end portion 850₂ are spirally wrapped around their respective struts 122 and crowns 120A until the first end portion 850₁ reaches the axial strut 126B₁ and the second end portion 850₂ reaches the axial strut 126B₂. In other words, the first end portion 850₁ is wrapped spirally around two struts 122 and a crowns 120A extending in the first direction D₁ (left in FIG. 8A) until the adjacent axial strut 126B in the first direction D₁ is reached (axial strut 126B₁). Similarly, the second end portion 850₂ is wrapped spirally around two struts 122 and a crown 120A extending in the second direction D₂ (right in FIG. 8A) until the adjacent axial strut 126B in the second direction D₂ is reached (axial strut 126B₂). The first end portion 850₁ and the second end portion 850₂ of the tissue strip 850 are then spirally wrapped around and down (towards the inflow end) the respective axial struts 126B₁, 126B₂ until the struts 112 extending from the respective inflow ends of the axial struts 126B₁, 126B₂ are reached, as illustrated in FIG. 8A.

Once the struts 112 are reached, the first end portion 850₁ and the second end portion 850₂ of the tissue strip 850 are spirally wrapped around their respective struts 112, crowns 110, and nodes 111 in a direction back towards the commissure post 126A to which the central or mid-portion 850c of the tissue strip 850 was secured, in this example the commissure post 126A₂. Thus, the first end portion 850₁ of the tissue strip 850 is wrapped in a spiral pattern in the second direction D₂ (back towards the commissure post 126A₂, to the right in FIG. 8A) along the strut 112 from the axial strut 126B₁ to the first node 111 adjacent to the axial strut 126B₁ in the second direction D₂. As illustrated in FIG. 8B, the first end portion 850₁ of the tissue strip 850 is wrapped around the struts 112 and passed through holes 804 in the skirt 195. In some embodiments, the holes 804 can be cut in the skirt 195 prior to attachment to the stent 102. In some embodiments, the holes 804 can be cut in the skirt 195 at the time of attachment of the tissue bumper 800. In some embodiments, the skirt 195 may have a serrated or wavy edge that forms holes 804 between the serrated edge of the skirt 195 and the struts 112 to which the skirt 195 is attached when the skirt 195 is attached to the stent 102. During wrapping, the holes 804 operate as a guide for wrapping the first end portion 850₁ and the second end portion 850₂ of the tissue strip 850. For example, as illustrated, the first end portion 850₁ of the tissue strip 850 can be wrapped around the first strut 112 two times, passing through two holes 804. The first end portion 850₁ of the tissue strip 850 at the junction of the axial strut 126B₁ and the strut 112 extending therefrom is wrapped from the exterior of the stent 102 to the interior of the stent 102 such that the tissue bumper 800 at the joint (intersection) of the strut 112 and axial strut 126B₁ is on the interior side of the stent 102 where abrasion may occur.

At the first node 111, the first end portion 850₁ of the tissue strip 850 is vertically wrapped to fully cover the first node 111 where abrasion may occur. Once the first node 111 is vertically wrapped, the first end portion 850₁ of the tissue strip 850 continues to be wrapped in the second direction D₂ in a spiral pattern (using the holes 804 as guides) along the next strut 112 at the outflow end of the inflow portion 108 until a crown 110 is reached. The first end portion 850₁ of the tissue strip 850 is wrapped past the crown (either covering the crown as shown in FIG. 8B or not covering the crown) and wrapped in a spiral pattern (using the holes 804 as guides) along the next strut 112 until the next node 111 is reached. The node 111 in this embodiment is adjacent to commissure post 126A₂ where the wrapping started. In particular, the node 111 is adjacent to commissure post 126A₂ in the first direction D₁. The second end portion 850₂ is similarly wrapped in the first direction D₁ around the struts 112, nodes 111, and crowns 110 from the axial strut 126B₂ until it reaches the node 111 adjacent the commissure post 126A₂ in the second direction.

In this embodiment, shown in FIG. 8A, when the first end portion 850₁ and the second end portion 850₂ of the tissue strip 850 reach the respective nodes 111 on either side of the commissure post 126A₂, the respective end portions 850₁ and 850₂ are wrapped from an exterior of the stent 102 to an interior of the stent 102 at the node 111 towards the outflow end of the stent 102, then under the strut 112 extending from the inflow end of the commissure post 126A₂ to an exterior side of the skirt 195. The first end portion 850₁ then runs along an exterior of the skirt 195 generally parallel to the strut extending from the second node 111 to the next adjacent node 111 in the first direction D₁ and towards the inflow end of the stent 102, as shown in FIGS. 8A and 8C. The end 802₁ of the first end portion 850₁ of the tissue strip 850 is generally axially aligned below (towards the inflow end) the commissure post 126A₂ where the wrapping began with the central portion 850c of the tissue strip 850. The end 802₂ of the second end portion 850₂ of the tissue strip 850 is similarly run along the exterior of the skirt 195 generally parallel to the corresponding strut 112 described above, but on the opposite site of the commissure post 126A₂. Thus, the end 802₂ of the second end portion 850₂ of the tissue strip 850 is generally axially aligned below (towards the inflow end) the commissure post 126A₂ where the wrapping began with the central portion 850c of the tissue strip 850. Further, this also puts the ends 802₁ and 802₂ of the tissue strip 850 adjacent to each other below (toward the inflow end) the commissure post 126A₂. These ends 802₁ and 802₂ may be secured to each other and/or to the skirt 195. If the ends 802₁ and 802₂ are secured to each other, the bumpers 800A, 800B, 900C may be considered a single or continuous tissue bumper 800.

Also in this embodiment, as illustrated in FIG. 8A, the first end portion 850₁ of a tissue strip 850 and the second end portion 850₂ of an adjacent tissue strip 850 are wrapped around the same axial strut 126B. Thus, for example, as shown in FIG. 8A, at the axial strut 126B₁, the first end portion 850₁ of the tissue strip 850 of the tissue bumper 800B and the second end portion 850₂ of the tissue strip 850 of the tissue bumper 800A are both wrapped down (towards the inflow end) of the axial strut 126B₁. This pattern is repeated for the other axial struts 126B and the other tissue bumpers 800. In some embodiments, the first end portion 850₁ and the second end portion 850₂ of adjacent tissue strips 850 can be wrapped, in the same direction, in a spiral pattern down the axial strut 126B such the first end portion 850₁ and the second end portion 850₂ of the adjacent tissue strips 850 do not overlap (e.g., a barber pole wrap). The barber pole wrap is described in further detail above with respect to FIGS. 3B-3C. In other embodiments, the tissue strips 850 can be wrapped using different patterns that allow the first end portion 850₁ and the second end portion 850₂ of the adjacent tissue strips 850 to overlap. For example, the first end portion 850₁ and the second end portion 850₂ of the adjacent tissue strips 850 can be wrapped in opposite directions such that the first end portion 850₁ and the second end portion 850₂ of the adjacent tissue strips 850 overlap (e.g., a weave wrap). In another example, one of the first end portion 850₁ or the second end portion 850₂ of the adjacent tissue strips 850 can be positioned vertically down the length of the interior surface of the axial strut 126B, and the other of the first end portion 850₁ or the second end portion 850₂ of the adjacent tissue strips 850 can be wrapped around the axial strut 126B, thereby securing the first end portion 850₁ or the second end portion 850₂ of the adjacent tissue strips 850 to the axial strut 126B (e.g., overlay wrap). The overlay wrap is further described below with respect to FIGS. 12A-12B.

As can be seen the tissue bumpers 800 of FIGS. 8A-8B are the same as the tissue bumpers 600 of FIGS. 6A-6C and the tissue bumpers 700 of FIGS. 7A-7C except at the ends of the tissue strips 850 as compared to the ends of the tissue strip 650 and the ends of the tissue strip 750.

In embodiments the tissue strip 850 may have a width in the range of 1 mm to 2.5 mm and may have a length in the range of 100 mm to 200 mm. However, this is not meant to be limiting and other dimensions may be used depending on the applications, such as, for example and not by way of limitation, the design of the stent, the size of the stent, and other factors.

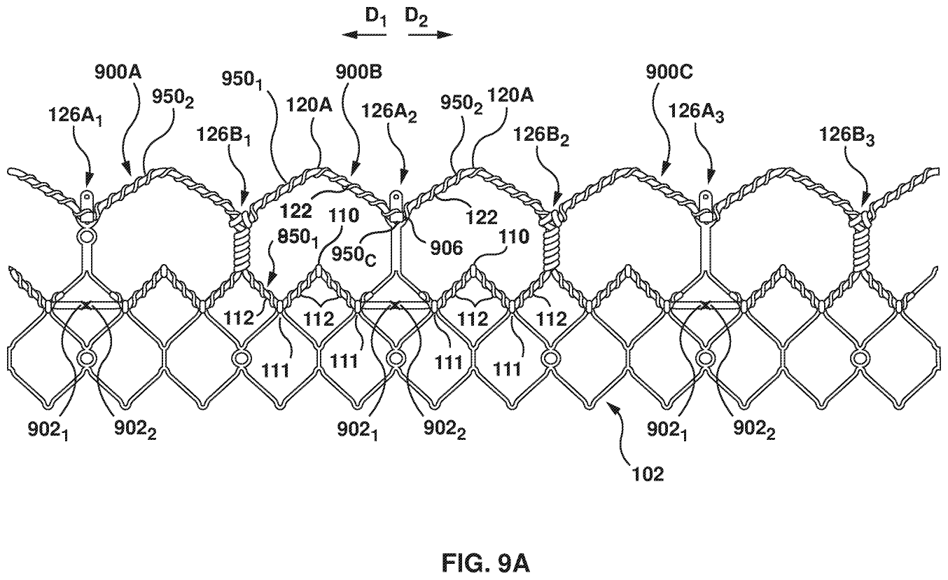

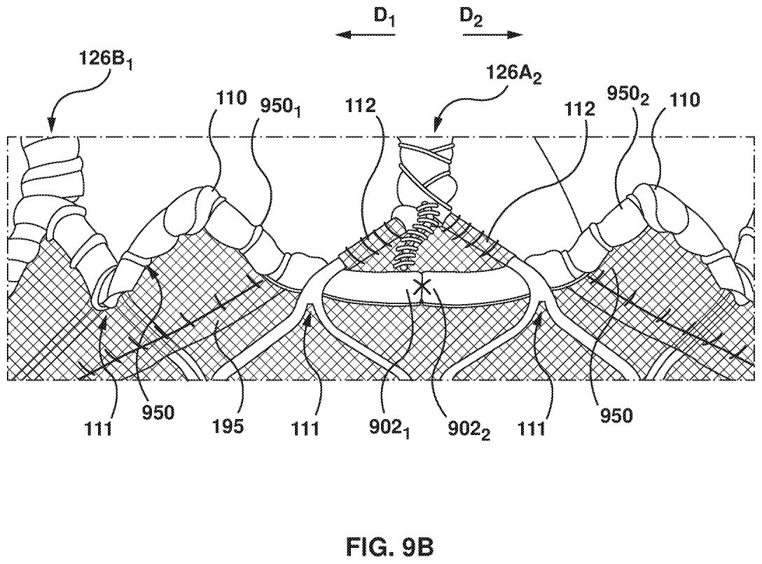

FIGS. 9A-9B illustrate an embodiment of tissue bumpers 900 attached to a stent, such as the stent 102. One skilled in the art will realize that FIGS. 9A-9C illustrate one example of tissue bumpers and that existing components illustrated in FIGS. 9A-9C may be removed and/or additional components may be added. Additionally, while not described in complete detail with reference to 9A-9C, the stent 102 can include any of the components described above with reference to FIGS. 1A-1C, and/or other features of stents know to those skilled in the art.

As illustrated in FIG. 9A, which is a simplified open, flat view of an example of the stent 102, the tissue bumpers 900 are positioned in areas of the stent 102 that may have a risk of leaflet contact with the stent 102. In particular, the tissue bumpers 900 are positioned on the struts 122 of the outflow portion 118, the axial struts 126B, and the struts 112 of the inflow portion 108 that surround the axial struts 126B. As illustrated, the stent 102 can include three tissue bumpers 900 that are each positioned on the two of three axial struts 126B, and the struts 112 and 122 that surround the three axial struts 126B of the stent 102. The three tissue bumpers 900 will be referred to herein individually as the tissue bumper 900A, 900B, and 900C when describing an individual tissue bumper for clarity. for In this embodiment, the tissue bumpers 900 can cover the entirety of the axial struts 126B, the struts 112 at the outflow end of the inflow portion 108, and the struts 122 that form the outflow portion 118.

In embodiments, each tissue bumper 900 may comprise a tissue strip 950, such as the tissue strip 250 described above with respect to FIG. 2D. In embodiments, the tissue strip 950 may be formed of natural materials, synthetic materials, and combinations thereof, as described above with reference to FIGS. 2A-2D. In embodiments, the tissue strip 950 can be secured to the stent 102 by wrapping or winding the tissue strip 950 around the commissure posts 126A, the axial struts 126B, the struts 122, the struts 112, and the nodes 111. In some embodiments, ends 902 of the tissue strip 950 can be secured to the struts 112 using sutures or stitches. In some embodiments, the ends 902 of the tissue strip 950 can be secured to the struts 112 by knotting the tissue strip 950.

The arrangement and attachment of one of the tissue bumpers 900 with respect to the stent 102 will now be described. It is understood that this arrangement and attachment may be used for each of the three tissue bumpers 900 shown in FIG. 9A. However, it is also understood that other embodiments described herein may be used in conjunction with the tissue bumper 900, or fewer than three tissue bumpers may be utilized. The tissue bumper 900B will be described herein, but as illustrated, the wrapping pattern/arrangement is the same for each of the three tissue bumpers 900, just that their locations are different. A central of mid-portion 950c of the tissue strip 950 forming the tissue bumper 900B is first secured to a commissure post 126A₂ at an intersection of the commissure post 126A₂ and the two struts 122 extending from the commissure post 126A₂ by forming an overlap knot 906 that secures the tissue strip 950 to the commissure post 126A₂. That is, the tissue strip 950 is wrapped around the commissure post 126A₂ at the intersection of the commissure post 126A₂ and the two struts 122, and knotted on the exterior of the stent 102 to secure the tissue strip 950 in place. For example, as illustrated in FIG. 9A, the central portion of mid-portion 950c of the tissue strip 950 can be aligned with an outflow end the commissure post 126A₂ at the intersection of the commissure post 126A₂ and the two struts 122. A first end portion 950₁ and a second end portion 950₂ of the tissue strip 950, disposed on opposite sides of the central portion 950c, are wrapped around the commissure post 126A₂ to form the overlap knot 906. A closer view detail of this wrapping can be seen in FIGS. 6B and 7B.

Once the knot 906 is formed, the first end portion 950₁ of the tissue strip 950 is wrapped spirally around the struts 122 and crowns 120A extending from the commissure post 126A₂ in a first direction D₁ (left in FIG. 9A), and the second end portion 950₂ of the tissue strip 950 is wrapped spirally around struts 122 extending from the commissure post 126A₂ in a second direction D₂ (right in FIG. 9A), opposite the first direction D₁. The first end portion 950₁ and the second end portion 950₂ are spirally wrapped around their respective struts 122 and crowns 120A until the first end portion 950₁ reaches the axial strut 126B₁ and the second end portion 950₂ reaches the axial strut 126B₂. In other words, the first end portion 950₁ is wrapped spirally around two struts 122 and a crown 120A extending in the first direction D₁ (left in FIG. 9A) until the adjacent axial strut 126B in the first direction D₁ is reached (axial strut 126B₁). Similarly, the second end portion 950₂ is wrapped spirally around two struts 122 and a crown 120A extending in the second direction D₂ (right in FIG. 9A) until the adjacent axial strut 126B in the second direction D₂ is reached (axial strut 126B₂). The first end portion 950₁ and the second end portion 950₂ of the tissue strip 950 are then spirally wrapped around and down (towards the inflow end) the respective axial struts 126B₁, 126B₂ until the struts 112 extending from the respective inflow ends of the axial struts 126B₁, 126B₂ are reached, as illustrated in FIG. 9A.

Once the struts 112 are reached, the first end portion 950₁ and the second end portion 950₂ of the tissue strip 950 are spirally wrapped around their respective struts 112, crowns 110, and nodes 111 in a direction back towards the commissure post 126A to which the central or mid-portion 950c of the tissue strip 950 was secured, in this example the commissure post 126A₂. Thus, the first end portion 950₁ of the tissue strip 950 is wrapped in a spiral pattern in the second direction D₂ (back towards the commissure post 126A₂, to the right in FIG. 9A) along the strut 112 from the axial strut 126B₁ to the first node 111 adjacent to the axial strut 126B₁ in the second direction D₂. As illustrated in FIG. 9B, the first end portion 950₁ of the tissue strip 950 is wrapped around the struts 112 and passed through holes 904 in the skirt 195. In some embodiments, the holes 904 can be cut in the skirt 195 prior to attachment to the stent 102. In some embodiments, the holes 904 can be cut in the skirt 195 at the time of attachment of the tissue bumper 900. In some embodiments, the skirt 195 may have a serrated or wavy edge that forms holes 904 between the serrated edge of the skirt 195 and the struts 112 to which the skirt 195 is attached when the skirt 195 is attached to the stent 102. During wrapping, the holes 904 operate as a guide for wrapping the first end portion 950₁ and the second end portion 950₂ of the tissue strip 950. For example, as illustrated, the first end portion 950₁ of the tissue strip 950 can be wrapped around the first strut 112 two times, passing through two holes 904. The first end portion 950₁ of the tissue strip 950 at the junction of the axial strut 126B₁ and the strut 112 extending therefrom is wrapped from the exterior of the stent 102 to the interior of the stent 102 such that the tissue bumper 900 at the joint (intersection) of the strut 112 and axial strut 126B₁ is on the interior side of the stent 102 where abrasion may occur.

As illustrated in FIG. 9B, at the first node 111, the first end portion 950₁ of the tissue strip 950 is vertically wrapped to fully cover the first node 111 where abrasion may occur. Once the first node 111 is vertically wrapped, the first end 950₁ of the tissue strip 950 continues to be wrapped in the second direction D₂ in a spiral pattern (using the holes 904 as guides) along the next strut 112 at the outflow end of the inflow portion 108 until a crown 110 is reached. The first end portion 850₁ is wrapped around the crown 110 and continues to be wrapped in a spiral pattern (using the holes 904 as guides) along the next strut 112 until the second node 111 is reached. The second node 111 in this embodiment is adjacent to commissure post 126A₂ where the wrapping started. In particular the second node 111 is adjacent to commissure post 126A₂ in the first direction D₁. The second end portion 950₂ is similarly wrapped in the first direction D₁ around the struts 112, nodes 111, and crowns 110 from the axial strut 126B₂ until it reaches the node 111 adjacent the commissure post 126A₂ in the second direction D₂.

In this embodiment, shown in FIGS. 9A and 9B, when the first end portion 950₁ and the second end portion 950₂ of the tissue strip 950 reach the respective nodes 111 on either side of the commissure post 126A₂, the respective end portions 950₁ and 950₂ are wrapped from an exterior of the stent 102 to an interior of the stent 102 over the node 111 towards the outflow end of the stent 102, then under the strut 112 extending from the inflow end of the commissure post 126A₂, then generally circumferentially along an exterior of the skirt 195. Each end 902₁, 902₂ of the tissue strip 850 ends generally axially aligned and below the commissure post 126A₂, as shown in FIG. 9B. In other words, the ends 9021, 902 generally face each other and are disposed at approximately the center of the cell 113 below (toward the inflow end) the commissure post 126A₂. These ends 902₁ and 902₂ may be secured to each other and/or to the skirt 195. If the ends 902₁ and 902₂ are secured to each other, the bumpers 900A, 900B, 900C may be considered a single or continuous tissue bumper 900.

Also in this embodiment, as illustrated in FIG. 9A, the first end portion 950₁ of a tissue strip 950 and the second end portion 950₂ of an adjacent tissue strip 950 are wrapped around the same axial strut 126B. Thus, for example, as shown in FIG. 9A, at the axial strut 126B₁, the first end portion 950₁ of the tissue strip 950 of the tissue bumper 900B and the second end portion 950₂ of the tissue strip 950 of the tissue bumper 900A are both wrapped down (towards the inflow end) of the axial strut 126B₁. This pattern is repeated for the other axial struts 126B and the other tissue bumpers 900. In some embodiments, the first end portion 950₁ and the second end portion 950₂ of adjacent tissue strips 950 can be wrapped, in the same direction, in a spiral pattern down the axial strut 126B such the first end portion 950₁ and the second end portion 950₂ of the adjacent tissue strips 950 do not overlap (e.g., a barber pole wrap). The barber pole wrap is described in further detail above with respect to FIGS. 3B-3C. In other embodiments, the tissue strips 950 can be wrapped using different patterns that allow the first end portion 950₁ and the second end portion 950₂ of the adjacent tissue strips 950 to overlap. For example, the first end portion 950₁ and the second end portion 950₂ of the adjacent tissue strips 950 can be wrapped in opposite directions such that the first end portion 950₁ and the second end portion 950₂ of the adjacent tissue strips 950 overlap (e.g., a weave wrap). In another example, one of the first end portion 950₁ or the second end portion 950₂ of the adjacent tissue strips 950 can be positioned vertically down the length of the interior surface of the axial strut 126B, and the other of the first end portion 950₁ or the second end portion 950₂ of the adjacent tissue strips 950 can be wrapped around the axial strut 126B, thereby securing the first end portion 950₁ or the second end portion 950₂ of the adjacent tissue strips 950 to the axial strut 126B (e.g., overlay wrap). The overlay wrap is further described below with respect to FIGS. 12A-12B.

As can be seen the tissue bumpers 900 of FIGS. 9A-9B are the same as the tissue bumpers 600 of FIGS. 6A-6C, the tissue bumpers 700 of FIGS. 7A-7C, and the tissue bumpers 800 of FIGS. 8A-8B, except at the ends of the tissue strips 950 as compared to the ends of the tissue strips 650, 750, 850.

In embodiments the tissue strip 950 may have a width in the range of 1 mm to 2.5 mm and may have a length in the range of 100 mm to 200 mm. However, this is not meant to be limiting and other dimensions may be used depending on the applications, such as, for example and not by way of limitation, the design of the stent, the size of the stent, and other factors.

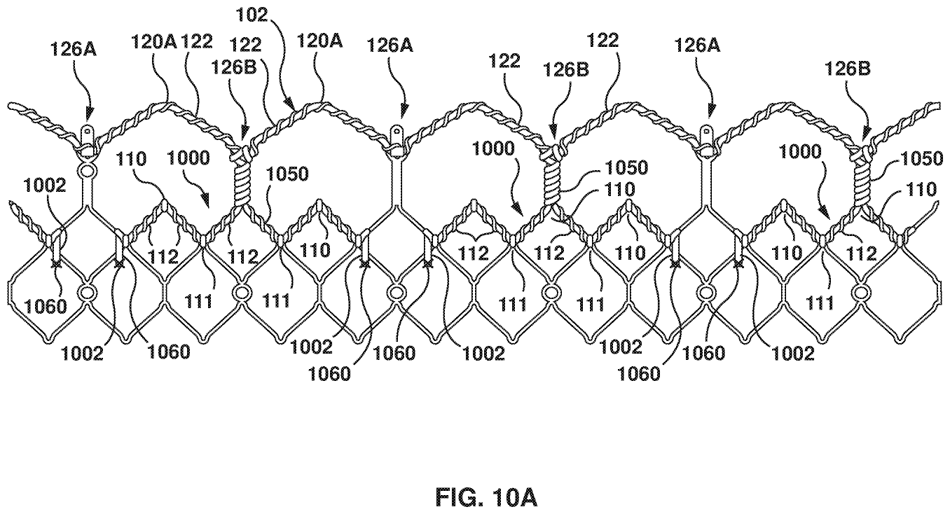

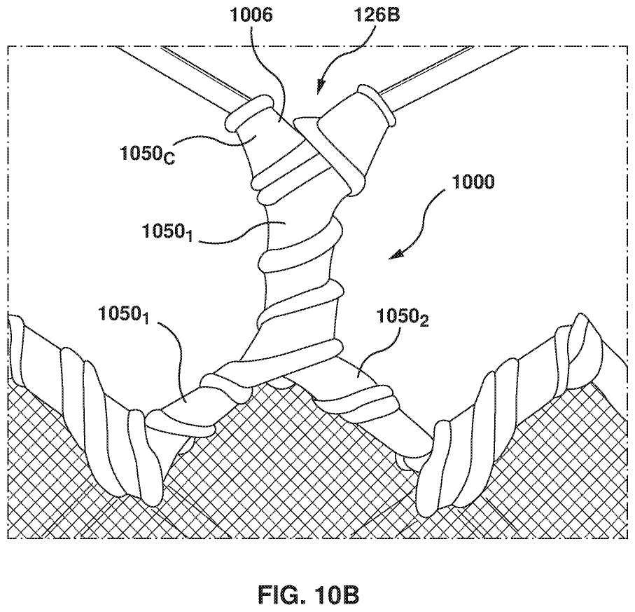

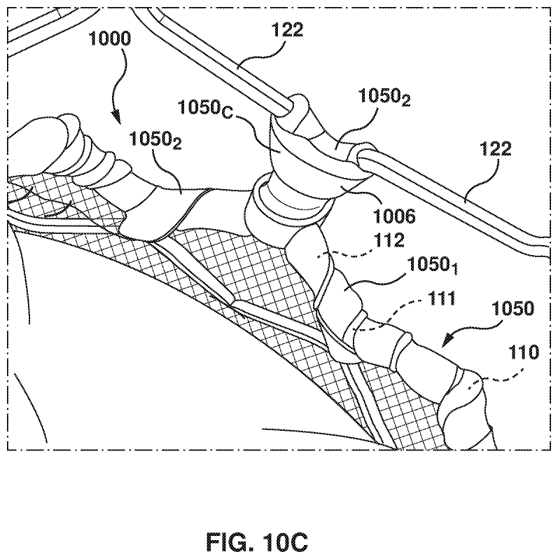

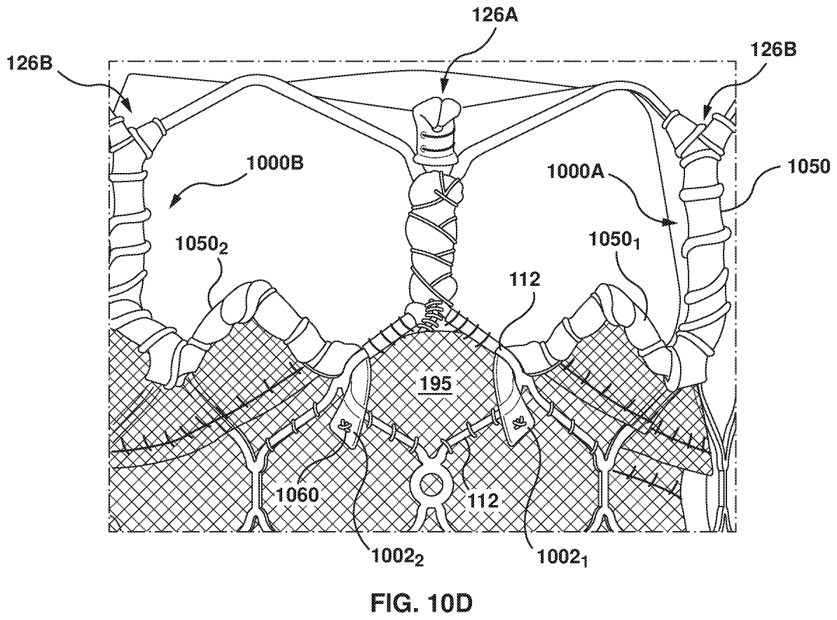

FIGS. 10A-10D illustrate tissue bumpers 1000 attached to a stent, such as the stent 102, according to an embodiment hereof. One skilled in the art will realize that FIGS. 10A-10D illustrate one example of tissue bumpers and that existing components illustrated in FIGS. 10A-10D may be removed and/or additional components may be added. Additionally, while not described in complete detail with reference to 10A-10D, the stent 102 can include any of the components described above with reference to FIGS. 1A-IC, and/or other features of stents know to those skilled in the art.

As illustrated in FIG. 10A, which is a simplified open, flat view of an example of the stent 102, each tissue bumper 1000 is positioned in areas of the stent 102 that may have a risk of leaflet contact with the stent 102. In particular, each tissue bumper 1000 is positioned on the axial struts 126B and on struts 112 of the inflow portion 108 that surround the axial struts 126B. As illustrated, the stent 102 can include three tissue bumpers 1000 that are positioned on the three axial struts 126B and the struts 112 that surround the three axial struts 126B of the stent 102. In this embodiment, the tissue bumper 1000 can cover the entire length of the three axial struts 126B.

In embodiments, each tissue bumper 1000 may comprise a tissue strip 1050, similar to the tissue strip 250 as described above with respect to FIG. 2D. In embodiments, the tissue strip 1050 may be formed of natural materials, synthetic materials, and combinations thereof, as described above with reference to FIGS. 2A-2D. In embodiments, the tissue strip 1050 can be secured to the stent 102 by wrapping or winding the tissue strip 1050 around the struts 112, the nodes 111, and the axial struts 126B. In some embodiments, ends 1002 of the tissue strip 1050 can be secured to the struts 112 using sutures or stitches. In some embodiments, the ends 1002 of the tissue strip 1050 can be secured to the struts 112 by knotting the tissue strip 1050.

As illustrated in FIGS. 10B-10C, the tissue strip 1050 is first secured to at an intersection of the axial strut 126B with struts 122 of the outflow portion 118 of the stent 102 by forming an overlapping knot 1006 that secures the tissue strip 1050 to the axial strut 126B and struts 122. That is, the tissue strip 1050 is wrapped around the intersection of the axial strut 126B and the struts 122, and knotted on the exterior of the stent 102 to secure the tissue strip 1050 in place. For example, as illustrated in FIG. 10B-10C, a central portion or mid-portion 1050c of the tissue strip 1050 can be aligned with an outflow end of the axial strut 126B. A first end portion 1050₁ of the tissue strip 1050 is wrapped around the intersection of the axial strut 126B and a first one of the struts 122 extending from the axial strut 126B. Then, a second end portion 1050₂ of the tissue strip 1050, opposite the first end portion 1050₁ with respect to the central portion 1050c, is wrapped around the intersection of the axial strut 126B and a second one of the struts 122 extending from the axial strut 126B, such the second end portion 10502₂ of the tissue strip 1050 overlaps the first end portion 1050₁ of the tissue strip 1050.

As illustrated in FIGS. 10B-10C, once the knot 1006 is formed, the second end portion 1050₂ of the tissue strip 1050 can be positioned vertically down the length of the interior surface of the axial strut 126B towards the inflow end of the stent 102, and the first end portion 1050₁ of the tissue strip 1050 can be wrapped around the axial strut 126B and the second end portion 1050₂ of the tissue strip 1050, thereby securing the second end portion 1050₂ of the tissue strip 1050 to the axial strut 126B (e.g., an overlay wrap). While FIGS. 10B-10C illustrate wrapping around the axial strut 126B in an overlapping pattern, the tissue strip 1050 can be wrapped down the axial strut 126B using different patterns that allow the first and second end portions 1050₁/1050₂ of the tissue strip 1050 to overlap. For example, the first and second end portions 1050₁/1050₂ of the tissue strip 1050 can be wrapped in opposite directions such that the first and second ends portions 1050₁/1050₂ of the tissue strip 1050 overlap (e.g., a weave wrap). In another example, after forming the knot 1006, the first and second end portions 1050₁/1050₂ of the tissue strip 1050 can be wrapped, in the same direction, in a spiral pattern down the axial strut 126B such that the first and second end portions 1050₁/1050₂ of the tissue strip 1050 do not overlap (e.g., a barber pole wrap). The barber pole wrap is illustrated in further detail above in FIGS. 3B-3C. Further, the overlay wrap is explained more detail with respect to FIGS. 12A and 12B below.

Once secured to the axial strut 126B, the first end portion 1050₁ of the tissue strip 1050 is wrapped in a spiral pattern along one of the struts 112 extending in a first direction from the axial strut 126B to the node 111. As illustrated in FIG. 10B, which is an enlarged view of a portion of the exterior of the stent, and FIG. 10C, which is an enlarged view of a portion of the interior of the stent 102, the first end portion 1050₁ of the tissue strip 1050 is wrapped around the strut 112 and may be passed through holes in the skirt 195. In some embodiments, the holes can be cut in the skirt 195 prior to attachment to the stent 102. In some embodiments, the holes can be cut in the skirt 195 at the time of attachment of the tissue strip 1050. In some embodiments, the skirt 195 may have a serrated or wavy edge that forms holes between the serrated edge of the skirt 195 and the struts 112 to which the skirt 195 is attached when the skirt 195 is attached to the stent 102. During wrapping, the holes operate as a guide for wrapping the tissue strip 1150. For example, as illustrated, the tissue strip 1150 can be wrapped around the strut 112 two times.

As illustrated in FIGS. 10B-10C, continuing with the first end portion 1050₁ of the tissue strip 1050, at the first node 111 in the first direction from the axial strut 126B, the tissue strip 1050 is vertically wrapped to fully cover the node 111 where abrasion may occur. Once the node 111 is vertically wrapped, the first end portion 1050₁ of the tissue strip 1050 continues to be wrapped in a spiral pattern in the first direction along the next strut 112 until a crown 110 is reached. In the embodiment as shown in FIGS. 10B-10D, the first end portion 1050₁ of the tissue strip 1050 is wrapped around the crown 110 and then in a spiral pattern in the first direction along the next strut 112 until a second node 111 is reached. However, as noted in other embodiments, the crown 110 in some embodiments may not be wrapped.

When the first end portion 1050₁ of the tissue strip 1050 reaches the second node 111 in the first direction from the axial strut 126B, the first end portion 1050₁ of the tissue strip 1050 is wrapped from the interior of the stent 102 to an exterior of the skirt 195. The first end portion 1050₁ of the tissue strip 1050 runs along an exterior of the skirt 195 generally axially or longitudinally towards the inflow end 106 of the stent 102. The first end portion 1050₁ of the tissue strip 1050 extends over the exterior of a strut 112 in the next adjacent row of struts and crowns in the direction of the inflow end of the stent 102. The struts 112 under and over which the first end portion 1050₁ extends form part of the cell below the commissure post 126A adjacent the axial strut 126B in the first direction, as best seen in FIG. 10D. The end 1002₁ of the first end portion 1050₁ of the tissue strip 1050 is then secured to the skirt 195 using sutures and/or knots 1060, as shown in FIGS. 10A and 10D. The wrapping described above with respect to the first end portion 1050₁ of the tissue strip 1050 in the first direction from the axial strut 126B is repeated for the second end portion 1050₂ of the tissue strip 1050 in the second direction. Further, in the embodiment shown, there are three tissue bumpers 1000 and thus three tissue strips 1050. Therefore, an end of a second end portion of a second one of the tissue strips 1050 that follows the wrapping pattern described above ends in the same cell of the same commissure post 126A as the first end portion 1050₁ described above, as shown in FIG. 10D. Thus, ends 1002₁ and 1002₂ of two different tissue strips 1050 are adjacent to each other on opposite sides and below each of the commissure posts 126A, as best seen in FIG. 10A. For example, as shown in FIG. 10D, a first end 1002₁ of a first end portion 1050₁ of a first tissue bumper 1000A is shown adjacent a second end 1002₂ of a second end portion 1050₂ of a second tissue bumper 1000B.

In embodiments the tissue strip 1050 may have a width in the range of 1 mm to 2.5 mm and may have a length in the range of 100 mm to 200 mm. However, this is not meant to be limiting and other dimensions may be used depending on the applications, such as, for example and not by way of limitation, the design of the stent, the size of the stent, and other factors.

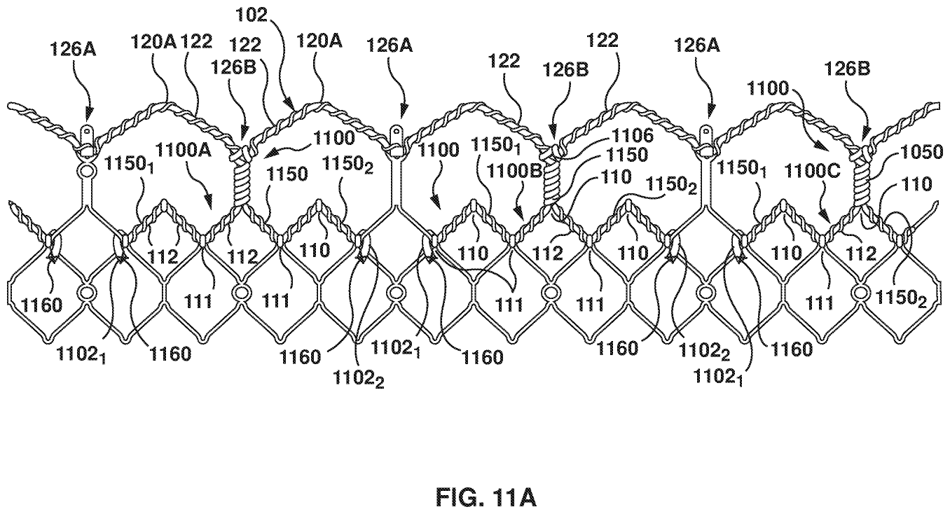

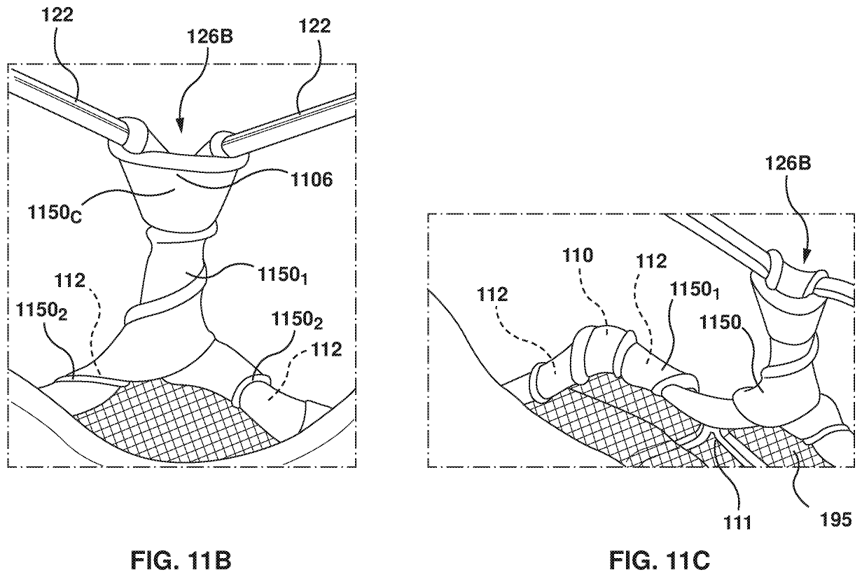

FIGS. 11A-11C illustrate tissue bumpers 1100 attached to a stent, such as the stent 102, according to an embodiment hereof. One skilled in the art will realize that FIGS. 11A-11C illustrate one example of tissue bumpers and that existing components illustrated in FIGS. 11A-11C may be removed and/or additional components may be added. Additionally, while not described in complete detail with reference to 11A-11C, the stent 102 can include any of the components described above with reference to FIGS. 1A-1C, and/or other features of stents know to those skilled in the art.

As illustrated in FIG. 11A, which is a simplified open, flat view of an example of the stent 102, each tissue bumper 1100 is positioned in areas of the stent 102 that may have a risk of leaflet contact with the stent 102. In particular, each tissue bumper 1100 is positioned on the axial struts 126B and on struts 112 of the inflow portion 108 that surround the axial struts 126B. As illustrated, the stent 102 can include three tissue bumpers 1000A, 1100B, 1100C that are positioned on the three axial struts 126B and the struts 112 that surround the three axial struts 126B of the stent 102. In this embodiment, each tissue bumper 1100 can cover the entire length of one of the three axial struts 126B.

In embodiments, each tissue bumper 1100 may comprise a tissue strip 1150, similar to the tissue strip 250 as described above with respect to FIG. 2D. In embodiments, the tissue strip 1150 may be formed of natural materials, synthetic materials, and combinations thereof, as described above with reference to FIGS. 2A-2D. In embodiments, the tissue strip 1150 can be secured to the stent 102 by wrapping or winding the tissue strip 1150 around the struts 112, the nodes 111, and the axial struts 126B. In some embodiments, ends 1102 of the tissue strip 1150 can be secured to the struts 112 and/or the skirt 195 using sutures or stitches. In some embodiments, the ends 1102 of the tissue strip 1150 can be secured to the struts 112 by knotting the tissue strip 1050.

As illustrated in FIGS. 11B-11C, the tissue strip 1150 is first secured to at an intersection of the axial strut 126B with struts 122 of the outflow portion 118 of the stent 102 by forming an overlapping knot 1106 that secures the tissue strip 1150 to the axial strut 126B and struts 122. That is, the tissue strip 1150 is wrapped around the intersection of the axial strut 126B and the struts 122, and knotted on the exterior of the stent 102 to secure the tissue strip 1150 in place. For example, as illustrated in FIG. 11B-11C, a central portion or mid-portion 1150c of the tissue strip 1150 can be aligned with an outflow end of the axial strut 126B. A first end portion 1150₁ of the tissue strip 1150 is wrapped around the intersection of the axial strut 126B and a first one of the struts 122 extending from the axial strut 126B. Then, a second end portion 1150₂ of the tissue strip 1050, opposite the first end portion 1050₁ with respect to the central portion 1150c, is wrapped around the intersection of the axial strut 126B and a second one of the struts 122 extending from the axial strut 126B, such the second end portion 1150₂ of the tissue strip 1150 overlaps the first end portion 1150₁ of the tissue strip 1050.

As illustrated in FIGS. 11B-11C, once the knot 1106 is formed, the second end portion 1150₂ of the tissue strip 1050 can be positioned vertically down the length of the interior surface of the axial strut 126B towards the inflow end of the stent 102, and the first end portion 1150₁ of the tissue strip 1050 can be wrapped around the axial strut 126B and the second end portion 1150₂ of the tissue strip 1150, thereby securing the second end portion 1150₂ of the tissue strip 1150 to the axial strut 126B (e.g., an overlay wrap). While FIGS. 11B-11C illustrate wrapping around the axial strut 126B in an overlapping pattern, the tissue strip 1150 can be wrapped down the axial strut 126B using different patterns that allow the first and second end portions 1150₁/1150₂ of the tissue strip 1150 to overlap. For example, the first and second end portions 1150₁/1150₂ of the tissue strip 1150 can be wrapped in opposite directions such that the first and second ends portions 1150₁/1150₂ of the tissue strip 1150 overlap (e.g., a weave wrap). In another example, after forming the knot 1106, the first and second end portions 1150₁/1150₂ of the tissue strip 1150 can be wrapped, in the same direction, in a spiral pattern down the axial strut 126B such that the first and second end portions 1150₁/1150₂ of the tissue strip 1150 do not overlap (e.g., a barber pole wrap). The barber pole wrap is illustrated in further detail above in FIGS. 3B-3C. Further, the overlay wrap is explained in more detail with respect to FIGS. 12A and 12B below.

Once secured to the axial strut 126B, the first end portion 1150₁ of the tissue strip 1150 is wrapped in a spiral pattern along one of the struts 112 extending in a first direction from the axial strut 126B to the node 111. As illustrated in FIGS. 11B and 11C, which are an enlarged views of a portion of the exterior of the stent 102, the first end portion 1150₁ of the tissue strip 1150 is wrapped around the strut 112. As discussed in other embodiments, but not specifically shown with respect to FIGS. 11A-11C, the tissue strip 1150 may be passed through holes in the skirt 195. In some embodiments, the holes can be cut in the skirt 195 prior to attachment to the stent 102. In some embodiments, the holes can be cut in the skirt 195 at the time of attachment of the tissue strip 1150. In some embodiments, the skirt 195 may have a serrated or wavy edge that forms holes between the serrated edge of the skirt 195 and the struts 112 to which the skirt 195 is attached when the skirt 195 is attached to the stent 102. If used, the holes operate as a guide for wrapping the tissue strip 1150. The first end portion 1150₁ of the tissue strip 1150 may be wrapped around the strut 112 two times before reaching the node 111 adjacent the axial strut 126B in the first direction, for example.

As illustrated in FIGS. 11A-11C, continuing with the first end portion 1150₁ of the tissue strip 1150, at the first node 111 in the first direction from the axial strut 126B, the tissue strip 1150 is extended circumferentially across the interior surface of the node 111. Thus, the exterior surface of the node 111 is not covered by the tissue strip 1150. Such a wrapping pattern protects the leaflets of the valve from abrasion as the leaflets are on the interior side of the stent 102, while enabling a smaller packing profile/diameter due to the exterior of the node 111 not being covered by the tissue strip. Once the first end portion 1150₁ of the tissue strip 1150 is extended circumferentially across the interior of the node 111, the first end portion 1150₁ of the tissue strip 1150 continues to be wrapped in a spiral pattern in the first direction along the next strut 112 until a crown 110 is reached. In the embodiment as shown in FIGS. 11A-11C, the first end portion 1150₁ of the tissue strip 1150 is wrapped around the crown 110 and then in a spiral pattern in the first direction along the next strut 112 until a second node 111 is reached. However, as noted in other embodiments, the crown 110 in some embodiments may not be wrapped.

When the first end portion 1150₁ of the tissue strip 1150 reaches the second node 111 in the first direction from the axial strut 126B, the first end portion 1150₁ of the tissue strip 1150 extends across the second node 111, is wrapped from under the next strut 112 from the interior of the stent 102 to an exterior of the skirt 195. The first end portion 1150₁ of the tissue strip 1150 runs along an exterior of the skirt 195 along an angle that is towards the inflow end and in the second direction, i.e., back towards the longitudinal axis of the axial strut 126B (i.e., an longitudinal extension of the axial strut 126B towards the inflow end). In other words, the end 1102₁ of the first end portion 1150₁ extends generally parallel to the struts 111, in the direction towards the inflow end and back towards to longitudinal axis of the axial strut 126B, as best shown in FIG. 11A. The first end portion 1150₁ of the tissue strip 1150 extends over the exterior of a strut 112 in the next adjacent row of struts and crowns in the direction of the inflow end of the stent 102. The struts 112 under and over which the first end portion 1150₁ extends form part of the cell below the commissure post 126A adjacent the axial strut 126B in the first direction, as best seen in FIG. 11A. The end 1102₁ of the first end portion 1150₁ of the tissue strip 1150 is then secured to the skirt 195 using sutures and/or knots 1160, as illustrated by the X-marks in FIG. 11A. The wrapping described above with respect to the first end portion 1150₁ of the tissue strip 1150 in the first direction from the axial strut 126B is repeated for the second end portion 1150₂ of the tissue strip 1150 in the second direction. Further, in the embodiment shown, there are three tissue bumpers 1100 and thus three tissue strips 1150. Therefore, an end of a second end portion of a second one of the tissue strips 1150 that follows the wrapping pattern described above ends in the same cell of the same commissure post 126A as the first end portion 1150₁ described above, as shown in FIG. 11A. Thus, ends 1102₁ and 1102₂ of two different tissue strips 1150 are adjacent to each other on opposite sides and below each of the commissure posts 126A, as best seen in FIG. 11A. For example, as shown in FIG. 11A, a second end 1102₂ of a second end portion 1150₂ of a first tissue bumper 1100A is shown adjacent a first end 1102₁ of a first end portion 1150₁ of a second tissue bumper 1100B.

In embodiments the tissue strip 1150 may have a width in the range of 1 mm to 2.5 mm and may have a length in the range of 100 mm to 200 mm. However, this is not meant to be limiting and other dimensions may be used depending on the applications, such as, for example and not by way of limitation, the design of the stent, the size of the stent, and other factors.

As can be seen, the embodiment of FIGS. 11A-11C is similar to the embodiment of FIGS. 10-10D except for the wrapping pattern at the nodes 111 and the wrapping pattern at the ends of the tissue strips. Further, the features of the embodiment of FIGS. 11A-11C may be used with any other embodiment herein. For example, and not by way of limitation, the wrapping pattern at the nodes 111 described above with respect to FIGS. 11A-11C can be used for wrapping the tissue strip for any of the other embodiments. Further, the ends of the tissue strips described with respect to FIGS. 11A-11C may be used with any of the other embodiments described herein.

FIGS. 12A-12B show in more detail the “overlay wrap” discussed above with respect to certain embodiments. In particular, FIG. 12A shows schematically a tissue strip 1250 of a tissue bumper 1200, which can be any of the tissue strips described above. FIG. 12A is a view from the interior of a stent, such as the stent 102, at one of the axial struts 126B. FIG. 12A shows a central portion 1250c of the tissue strip 1250 placed at the outflow end of the axial strut 126B to the interior side of the axial strut 126B. A first end portion 1250₁ of the tissue strip 1250 is extended from the interior to the exterior of a first outflow strut 122₁ as the first end portion 1250₁ extends from the inflow side to the outflow side of the first outflow strut 122₁. The first end portion 1250₁ is wrapped around the first outflow strut 122₁ and extended towards the inflow end of the stent 102 through an opening 1270 between the central portion 1250c and the axial strut 126B. Similarly, a second end portion 1250₂ of the tissue strip 1250 is extended from the interior to the exterior of a second outflow strut 122₂ that extends from an opposite circumferential side of the axial strut 126B as the first outflow strut 122₁ as the second end portion 1250₂ extends from the inflow side to the outflow side of second outflow strut 122₂. The second end portion 1250₂ is wrapped around the second outflow strut 122₂ and extended towards the inflow end of the stent 102 through the opening 1270 between the central portion 1250c and the axial strut 126B. These steps form an overlapping knot 1206 securing the tissue strip 1250 to the axial strut 126B.

The second end portion 1250₂ of the tissue strip 1250 is then extended along the interior surface of the axial strut 126B from the outflow end to the inflow end of the axial strut 126B. The first end portion 1250₁ of the tissue strip 1250 is then wrapped spirally around the axial strut 126B and the second end portion 1250₂ from the outflow end to the inflow end of the axial strut 126B, as shown in FIG. 12A. This overlay wrap results in two layers of the tissue strip 1250 on an interior side of the axial strut 126B and a single layer of the tissue strip 1250 on the exterior side of the axial strut 126B, as shown in FIG. 12B.

The first end portion 1250₁ is then be wrapped around a strut 112 on a first direction from the axial strut 126B and the second end portion 1250₂ is wrapped around another strut 112 in a second direction from the axial strut 126B, as described in several embodiments above. The overlay wrap described with respect to FIGS. 12A-12B can be used with any of the embodiments described above.

The term “generally” when used herein such as in “generally longitudinal” or “generally axial” or “generally circumferentially” or “generally parallel” or similar language means within 10 degrees of longitudinal or axial or circumferential or parallel.

While the components of the prosthetic heart valve 100 are described above with relative terms “first,” “second,” “proximal,” and “distal,” one skilled in the art will realize that the use of these terms is intended only to identify components of the prosthetic heart valve 100 and do not define any preferred or ordinal arrangement of the components of prosthetic heart valve 100.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a medical device.

1. A prosthetic heart valve that is radially collapsible to a collapsed configuration and radially expandable to an expanded configuration, the prosthetic heart valve comprising:
an annular stent having a longitudinal axis extending between an inflow end of the stent and an outflow end of the annular stent, the annular stent comprising:
an inflow portion comprising a plurality of rows of angled inflow struts,
an outflow portion comprising a row of angled outflow struts, and
a transition portion comprising an axial strut and a commissure post;
a prosthetic valve including a leaflet structure positioned within the stent and secured to the at least one commissure post; and
a tissue bumper configured to reduce abrasion caused by contact by the leaflet structure with the annular stent, the tissue bumper comprising a tissue strip wrapped around at least one angled outflow strut in the row of angled outflow struts, the axial strut, and a portion of a first row of angled struts of the plurality of rows of angled inflow struts.

2. The prosthetic heart valve of claim 1, wherein:
a center portion of the tissue strip is wrapped around an outflow end of the commissure post;
a first end portion the tissue strip extends from the commissure post in a first direction and is wrapped around outflow struts of the row of angled outflow struts to the axial strut;
the first end portion extends along and is wrapped around the axial strut toward the inflow end of the annular stent; and
the first end portion extends in a second direction opposite the first direction from an inflow end of the axial strut and wraps around angled inflow struts of the first row of angled inflow struts.

3. The prosthetic heart valve of claim 2, wherein the first end portion extends along at least two angled inflow struts of the first row of angled inflow struts.

4. The prosthetic heart valve of claim 3, wherein:
a second end portion the tissue strip extends from the commissure post in the second direction and is wrapped around outflow struts of the row of angled outflow struts to a second axial strut disposed on an opposite side of the commissure post as the axial strut;
the second end portion extends along and is wrapped around the second axial strut toward the inflow end of the annular stent; and
the second end portion extends in the first direction from an inflow end of the second axial strut and wraps around angled inflow struts of the first row of angled inflow struts.

5. The prosthetic heart valve of claim 2, wherein the first end portion of the tissue strip extends longitudinally towards the inflow end of the annular stent to a first end of the tissue strip.

6. The prosthetic heart valve of claim 5, wherein:
a second end portion the tissue strip extends from the commissure post in the second direction and is wrapped around outflow struts of the row of angled outflow struts to a second axial strut disposed on an opposite side of the commissure post as the axial strut;
the second end portion extends along and is wrapped around the second axial strut toward the inflow end of the annular stent;
the second end portion extends in the first direction from an inflow end of the second axial strut and wraps around angled inflow struts of the first row of angled inflow struts; and
the second end portion of the tissue strip extends longitudinally towards the inflow end of the annular stent to a second end of the tissue strip.

7. The prosthetic heart valve of claim 6, further comprising a skirt coupled to the inflow portion of the annular stent, wherein the first and second ends of the tissue bumper are coupled to an exterior surface of the skirt.

8. The prosthetic heart valve of claim 3, wherein the first end portion of the tissue strip extends generally parallel to an angled inflow strut of a second row of the plurality of rows of angled inflow struts to a first end of the tissue strip generally axially aligned with the commissure post.

9. The heart valve prosthesis of claim 8, wherein:
a second end portion the tissue strip extends from the commissure post in the second direction and is wrapped around outflow struts of the row of angled outflow struts to a second axial strut disposed on an opposite side of the commissure post as the axial strut; the second end portion extends along and is wrapped around the second axial strut toward the inflow end of the annular stent;
the second end portion extends in the first direction from an inflow end of the second axial strut and wraps around angled inflow struts of the first row of angled inflow struts; and the second end portion of the tissue strip extends generally parallel to an angled inflow strut of a second row of the plurality of rows of angled inflow struts to a second end of the tissue strip generally axially aligned with the commissure post.

10. The prosthetic heart valve of claim 9, further comprising a skirt coupled to the inflow portion of the annular stent, wherein the first and second ends of the tissue strip are coupled to an exterior surface of the skirt.

11. The prosthetic heart valve of claim 9, wherein the first and second ends of the tissue strip are attached to each other.

12. The prosthetic heart valve of claim 3, wherein the first end portion of the tissue strip extends generally circumferentially to a first end disposed generally at a center of a cell defined by the first row and a second row of the plurality an angled inflow struts, wherein the center of the cell is axially aligned with the commissure post.

13. The heart valve prosthesis of claim 12, wherein:
a second end portion the tissue strip extends from the commissure post in the second direction and is wrapped around outflow struts of the row of angled outflow struts to a second axial strut disposed on an opposite side of the commissure post as the axial strut;
the second end portion extends along and is wrapped around the second axial strut toward the inflow end of the annular stent;
the second end portion extends in the first direction from an inflow end of the second axial strut and wraps around angled inflow struts of the first row of angled inflow struts; and
the second end portion of the tissue strip extends generally circumferentially to a first end disposed generally at the center of the cell.

14. The prosthetic heart valve of claim 13, further comprising a skirt coupled to the inflow portion of the annular stent, wherein the first and second ends of the tissue strip are coupled to an exterior surface of the skirt.

15. The prosthetic heart valve of claim 13, wherein the first and second ends of the tissue strip are attached to each other.

16. The prosthetic heart valve of claim 4, wherein the annular stent further comprises a second commissure post and a second tissue bumper comprising a second tissue strip, wherein a first end portion of the second tissue strip overlaps with the second end portion of the tissue strip of the tissue bumper along the second axial strut.

17. A prosthetic heart valve comprising:
an annular stent having a longitudinal axis extending between an inflow end of the stent and an outflow end of the annular stent, the annular stent comprising:
an inflow portion comprising a plurality of rows of angled inflow struts,
an outflow portion comprising a row of angled outflow struts, and
a transition portion comprising three commissure posts and three frame portions, each of the commissure posts having a proximal end coupled to the inflow portion and a distal end coupled to the outflow portion extending from the inflow portion to the outflow portion, and each frame portion having first end coupled to the inflow portion and a second end coupled to the outflow portion, wherein the commissure posts and the frame portions alternate around a circumference of the annular stent;
a prosthetic valve including a leaflet structure positioned within the stent and secured to the commissure posts; and
protective bumpers configured to reduce abrasion caused by contact by the leaflet structure with the annular stent, each protective bumper covering a respective frame portion of the three frame portions.

18. The prosthetic heart valve of claim 17, wherein each of the frame portions is directly connected to two adjacent commissure posts by two angled outflow struts of the row of angled outflow struts, the two angled outflow struts extending in opposite directions from the frame portion.

19. The prosthetic heart valve of claim 17, further comprising a skirt coupled to the inflow portion of the annular stent, wherein the skirt and the protective bumpers are separate pieces of material.

20. The prosthetic heart valve of claim 17, wherein the bumpers are tissue bumpers.

21. The prosthetic heart valve of claim 20, wherein the tissue bumpers comprise pericardium tissue.

22. The prosthetic heart valve of claim 17, wherein each bumper comprises a strip of material wrapped around the respective frame portion.

23. The prosthetic heart valve of claim 22, wherein each the strip of material further wraps around at least one angled outflow strut in the row of angled outflow struts and/or a portion of a first row of angled struts of the plurality of rows of angled inflow struts.
